(19) United States
(12) Patent Application Publication
(10) Pub. No.: US 2005/0130172 A1
(43) Pub. Date: Jun. 16, 2005

(54) Identification and verification of methylation marker sequences

(75) Inventors: Chris Beard, Concord, CA (US); Chris Burgess, Westwood, MA (US); Allison Gannon, Walnut Creek, CA (US); Jeanne Harvey, Livermore, CA (US); John F. Lechner, Richmond, CA (US); Zheng Li, Hayward, CA (US)

Correspondence Address: PALMER & DODGE, LLP;PAULA CAMPBELL EVANS, 111 HUNTINGTON AVENUE, BOSTON, MA, 02199, US

(73) Assignee: Bayer Corporation

(21) Appl. No.: 10/765,790

(22) Filed: Jan. 27, 2004

Related U.S. Application Data
(63) Continuation-in-part of application No. 10/737,082, filed on Dec. 16, 2003.

(51) Int. Cl. C12Q 1/68; G06F 19/00; G01N 33/48; G01N 33/50

(52) U.S. Cl. 435/6; 702/20

(57) ABSTRACT

The present invention relates to methods for identifying among the genes that are down-regulated in cells or tissues having disease including cancer, the CpG sites within the CpG islands of said genes, wherein the identified CpG sites show great potential for diagnostic utility. In another aspect, the present invention also provides methods of using the selected CpG sites for purposes of diagnosis, prognosis, staging, assessing or monitoring the therapy of or recovery from a disease such as cancer.

### RELATED APPLICATION

This application is a continuation-in-part of application Ser. No. 10/737,082 filed on Dec. 16, 2003.

### SEQUENCE LISTING

This application includes a sequence listing submitted on compact disc in triplicate (three) compact discs: Computer Readable Copy (disk 1), Copy 1 (disk 2) and Copy 2 (disk 3), the contents of which are hereby incorporated by reference in its entirety. All three compact discs contain identical sequences. The following information is identical for each CD-ROM submitted: Machine Format: IBM-PC; Operating System: MS-Windows;

| | | DATE |
|---|---|---|
| FILE NAME | SIZE | OF CREATION |
| SEQUENCE_LISTING-Bayer-2035 | 9,554 KB | Jan. 26, 2004 |

The information on each CD-ROM is incorporated herein by reference in its entirety.

### FIELD OF THE INVENTION

The present invention generally relates to methods for identifying the CpG sites that show great potential for diagnostic utility. Furthermore, the present invention relates to methods of using the identified CpG sites for diagnosis, prognosis, and staging of a disease, and assessment of therapy in a subject.

### BACKGROUND OF THE INVENTION

In mammals, DNA methylation usually occurs at cytosines located 5′ of guanines, known as CpG dinucleotides. DNA (cytosine-5)-methyltransferase (DNA-Mtase) catalyzes this reaction by adding a methyl group from S-adenosyl-L-methionine to the fifth carbon position of the cytosine. Chiang, P K, et al., “S-adenosylmethionine and methylation,” FASEB J., 10: 471-480 (1996). Most cytosines within CpG dinucleotides are methylated in the human genome, but some remain unmethylated in specific GC-rich areas. These areas are called CpG islands. Antequera, F. et al., “High levels of de novo methylation and altered chromatin structure at CpG islands in cell lines,” Cell, 62: 503-514 (1990). CpG islands are typically between 0.2 to about 1 kb in length and are located upstream of many housekeeping and tissue-specific genes, but may also extend into gene coding regions. Antequera, F. et al., “High levels of de novo methylation and altered chromatin structure at CpG islands in cell lines,” Cell, 62: 503-514 (1990).

DNA methylation is a heritable, reversible, and epigenetic change; it has the potential to alter gene expression, which has profound developmental and genetic consequences. DNA methylation is known to play a role in regulating gene expression during cell development. This epigenetic event frequently is associated with transcriptional silencing of imprinted genes, some repetitive elements and genes on the inactive X chromosome. Li, E. et al, “Role for DNA methylation in genomic imprinting,” Nature, 366: 362-365 (1993); Singer-Sam, J. and Riggs, A D, X chromosome inactivation and DNA methylation; Jost, J. P. and Saluz, H. P. (eds), DNA Methylation: molecular Biology and Biological Significance, Birkhaeuser Verlag, Basel, Switzerland, pp. 358-384 (1993). In neoplastic cells, it has been observed that the normally unmethylated CpG islands can become aberrantly methylated, or hypermethylated. Jones, P A, “DNA methylation errors and cancer,” Cancer Res., 56:2463-2467 (1996).

Aberrantly methylated cytosine at CpG dinucleotides is a widespread phenomenon in cancer. Jones, P A and Laird, P W, “Cancer epigenetics comes of age,” Nat. Genet. 21: 163-167 (1999). As a result of CpG island hypermethylation, chromatin structure in the promoter can be altered, preventing normal interaction with the transcriptional machinery. Baylin, SB, et al. “Alterations in DNA methylation: A fundamental aspect of neoplasia,” in Advances in cancer research (eds. G. F. Vande Woude and G. Klein), vol. 72: 141-196 (1998), Academic Press, San Diego, Calif. When this occurs in genes critical to growth inhibition, the resulting silencing of transcription could promote tumor progression. In addition, promoter CpG island hypermethylation has been shown to be a common mechanism for transcriptional inactivation of classic tumor suppressor genes and genes important for cell cycle regulation, and DNA mismatch repair. Methylation of cytosine, therefore, plays a significant role in control of gene expression, and a change in the methylation pattern or status is likely to cause disease.

### SUMMARY OF THE INVENTION

The present invention relates to methods for identifying among nucleic acid sequences that are down-regulated in cells or tissues having disease, including cancer, these CpG sites within the CpG islands of said nucleic acid sequences, the methylation status or state of which is indicative of the presence or stage of the disease. The invention further pertains to the use of such sequences as biomarkers for the presence or stage of the disease, or as indicators of the efficacy of therapy.

In one aspect, the present invention pertains to identification of down-regulated (under-expressed) nucleic acid marker sequences in a biological sample from a patient having or suspected of having a disease or disorder, such as cancer or a pre-malignant condition. In general, the method of identifying the nucleic acid marker sequences includes (1) providing a pool of target nucleic acids preferably derived from both disease and normal cells and/or tissues and preferably comprising RNA transcripts of the target markers derived from the RNA transcripts; (2) hybridizing the nucleic acid samples to one or more probes; and (3) detecting the hybridized nucleic acids and determining the expression levels derived from the diseased cells/tissues relative to the expression levels of the same nucleic acids from normal cells and/or tissues. Various conventional methods known in the art may be employed to identify the nucleic acid marker sequences that are down-regulated in a disease, especially cancer. In one embodiment, microarrays such as DNA arrays are employed in the method.

The present invention further provides nucleic acid marker sequences that are down-regulated in disease, including cancer or tumor, identified using the above method. The present invention further provides polynucleotides which are at least about 85%, at least about 90%, or more preferably at least about 95% identical to the sequences of the RNA transcripts or cDNAs of the down-regulated nucleic acid marker sequences, and polypeptides encoded by the nucleic acid marker sequences.

In another aspect, the present invention pertains to the identification of CpG islands on the down-regulated nucleic acid marker sequences. CpG islands are defined to be short nucleic acid sequences greater than 200 bp in length, with a GC content greater than 0.5 and an observed to expected ratio based on GC content greater than 0.6. See Gardiner-Garden and Frommer, “CpG islands in vertebrate genomes,” J. Mol. Biol. 196(2): 261-282 (1987). CpG islands may be identified by any method known in the art using the Gardiner-Garden and Frommer definition. The present invention further provides the nucleic acid sequences containing the CpG islands within the promoter-first exon region of the genes encoded by the nucleic acid marker sequences that are down-regulated in disease such as cancerous or premalignant cells or tissues.

In another aspect, the present invention pertains to determining whether the candidate CpG sites within the CpG islands of the down-regulated marker sequences are methylated in diseased cells or tissues. This can be performed by using methylation assays capable of determining differential methylation levels within CpG sites between diseased cells or tissues and normal cells or tissues. Methylation-specific assays useful for this purpose include, for example, methylation-specific PCR, bisulfite genomic sequencing methods, methylation-specific primer extension methods, and all other methods known in the art, and with high throughput or microarrays.

In another aspect, the present invention pertains to selection of CpG sites within the CpG islands of the down-regulated marker sequences that have the greatest potential in diagnostic, prognostic and therapeutic assays for detecting a disease. Generally, the selection comprises the steps of (1) determining the functional recovery of the down-regulated marker sequences containing the methylated CpG sites after demethylation treatment, and (2) validating the CpG sites on the nucleic acid marker sequences in clinical samples.

In step (1), the nucleic acid sequences containing the methylated CpG sites are further determined for functional recovery after demethylation treatment. Functional recovery after demethylation treatment would result in a significant increase in the nucleic acid expression levels of the nucleic acid sequences containing the CpG sites after the demethylation treatment. The term “significant increase in the nucleic acid expression levels” as used herein, refers to an increase in nucleic acid expression levels by at least about 10%, preferably at least about 15%, about 25%, about 30%, about 40%, about 50%, about 65%, about 75%, about 85%, about 90%, about 95% or greater. In another embodiment, functional recovery after demethylation treatment would also result in a significant increase in the levels of the proteins encoded by the down-regulated marker sequences containing the CpG sites after demethylation treatment. The term “significant increase in the levels of the proteins” as used herein, refers to an increase in protein levels by at least about 15%, preferably at least about 25%, 35%, 50%, or greater. In yet another embodiment, functional recovery after demethylation treatment would also mean a significant restoration of functional phenotypes associated with the functionality of the proteins encoded by the down-regulated marker sequences containing methylated CpG sites after the demethylation treatment.

In step (2), the validation of the CpG sites selected by methods in step (1) comprises determining correlation of the methylation of the CpG sites with a disease in clinical samples. Preferably, the correlation is determined by detecting the methylation of the CpG sites in clinical samples obtained from a subject afflicted with or suspected of having a disease to be detected compared to that in a normal, disease-free sample. A good correlation between the methylation at a specific CpG site and a disease could mean that the said specific CpG site is hypermethylated in samples obtained from a subject afflicted with or suspected of having disease compared to that in normal, disease-free samples. The CpG sites that show a significant increase in methylation in samples obtained from a subject afflicted with or suspected of having disease compared to that in normal, disease-free samples, are preferably selected. Preferably, the increase in methylation of the CpG sites in the disease sample is by at least about 1.5 fold, more preferably at least about 2 fold over that in a normal sample.

In addition, a good correlation between the methylation at a specific CpG site and a disease could also mean that the degree of methylation at the CpG site shows distinct differences at different stages of a disease.

A good correlation could also encompass the relationship between multiple CpG sites on a single nucleic acid marker sequence and a disease. For example, for one specific disease to be assayed, the methylation at one or more CpG sites on a single nucleic acid marker sequence could either increase or decrease as the disease progresses to advanced stages. Alternatively, either increased number of or decreased number of CpG sites on a single nucleic acid marker sequence could be methylated as the disease progresses to advanced stages.

The nucleic acid sequences whose CpG sites show good correlation between the methylation of the CpG sites and disease in clinical samples, are preferably selected for uses in diagnosis, prognosis, staging, monitoring, and therapeutic treatment of a disease. Preferably, diagnosis, prognosis, staging, monitoring, and therapeutic treatment of a disease are performed by detecting the methylation of the CpG sites on the nucleic acid sequences from samples obtained from a subject having or suspected of having a disease to be detected.

As a result of the selection, the selected nucleic acid sequences should contain the CpG sites showing a significant increase in methylation in samples from tissues or cells afflicted with or suspected of disease compared to samples from normal tissues or cells, and exhibit functional recovery after demethylation treatment.

In another aspect, the present invention provides methods of using the identified CpG sites on the selected nucleic acid marker sequences for purposes of diagnosis, prognosis, staging, assessing or monitoring the therapy of or recovery from a disease such as cancer including colon cancer, breast cancer, lung cancer, head and neck cancer, liver cancer, and leukemia, neurodegenerative diseases such as Huntington's disease, Alzheimer's disease, Rett syndrome, hypertension, etc.

The present invention provides methods for detecting the presence, or predisposition of a disease such as cancer, by detecting methylation levels of one or more selected CpG sites within one or more down-regulated marker sequences, wherein the methylation of the CpG sites corresponds to a disease. Preferably, the CpG sites are the ones selected by the methods of the present invention. Particularly, the method of detecting, or diagnosing a disease in a subject, comprises:
(a) determining the degree of methylation of one or more CpG sites on nucleic acid sequences in a biological sample obtained from the subject;
(b) determining the presence of, predisposition to, or stage of the disease in the subject based on the degree of methylation.

The present invention also provides methods for determining disease prognosis and stage based on examining the methylation levels of the selected CpG sites within one or more down-regulated marker sequences, wherein the different methylation levels of the CpG sites correspond to different stages of a disease. Particularly, the method of monitoring the onset, progression, or regression of a disease in a subject, comprises:
(a) detecting in a biological sample of the subject at a first point in time, methylation levels of one or more CpG sites, wherein the CpG sites are differentially methylated at different stages of the disease;
(b) repeating step (a) at a subsequent point in time; and
(c) comparing the methylation levels of the CpG sites in step (a) and (b), wherein a change in the methylation levels is indicative of disease progression in the subject.

The present invention also provides methods that permit the assessment and/or monitoring of patients who will be likely to benefit from both traditional and non-traditional treatments and therapies for disease such as, particularly colon cancer. The method for determining the efficacy of a test compound for ameliorating or inhibiting a disease in a subject comprises:
(a) detecting in a first biological sample of the subject, methylation levels of one or more CpG sites, wherein the sample has not been exposed to the test compound, and wherein the CpG sites are methylated in the disease;
(b) detecting in a second biological sample of the subject, methylation levels of the same CpG sites, wherein the sample has been exposed to the test compound; and
(c) comparing the methylation levels of the CpG sites in step (a) and (b), wherein a decrease in methylation after the sample has been exposed to the test compound, is indicative of the efficacy of the test compound.

The present invention also provides a kit for practicing the uses of the selected CpG sites on the nucleic acid marker sequences in diagnosis, prognosis, staging, and monitoring of the therapy. The kit may comprise a bisulfite-containing reagent that modifies the unmethylated cytosine, as well as oligonucleotides involved in detecting the methylation of one or more specific CpG sites on a specific nucleic acid marker sequence, wherein said detection of the methylation comprises one or more of the following techniques: methylation-specific PCR, bisulfite genomic sequencing methods, methylation-specific primer extension methods, and all other methods known in the art, and with high throughput or microarrays.

A kit may also comprise a control/reference value or a set of control/reference values indicating normal and various clinical progression stages of a disease. In one embodiment, the control/reference value or a set of control/reference values is indicative of various clinical progression stages of cancer. In a preferred embodiment, the control/reference value or a set of control/reference values is indicative of various clinical progression stages of colon cancer. Moreover, a kit may also comprise positive controls, and/or negative controls for comparison with the test sample. A negative control may comprise a sample that does not have any nucleic acid marker sequences. A positive control may comprise various degrees of methylation at one or more specific CpG sites. A kit may further comprise instructions for carrying out and evaluating the results.

### DETAILED DESCRIPTION OF THE INVENTION

I Definitions

As used herein, the term “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). “A biological sample” further refers to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. Most often, the sample has been removed from an animal, but the term “biological sample” can also refer to cells or tissue analyzed in vivo, i.e., without removal from animal. Typically, a “biological sample” will contain cells from the animal, but the term can also refer to non-cellular biological material, such as non-cellular fractions of blood, saliva, or urine, that can be used to measure the cancer-associated polynucleotide or polypeptide levels. “A biological sample” further refers to a medium, such as a nutrient broth or gel in which an organism has been propagated, which contains cellular components, such as proteins or nucleic acid molecules.

As used herein, the term “biomarker” or “marker” refers to a biological molecule, e.g., a nucleic acid, peptide, hormone, etc., whose presence or concentration can be detected and correlated with a known condition, such as a disease state. The term “biomarker” also refers to any molecule derived from a gene, e.g., a transcript of the gene or a fragment thereof, a sense (coding) or antisense (non-coding) probe sequence derived from the gene, or a full length or partial length translation product of the gene or an antibody thereto, which can be used to monitor a condition, disorder, disease, or the status in the progression of a process.

As used herein, the term “a clinical sample” refers to a sample as defined herein from a medical patient.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, the term “a polynucleotide primer/probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.) or sugar moiety. In addition, the bases in a primer/probe may be joined by a linkage other than a phosphodiester bond, so long as it does not interfere with hybridization. Thus, for example, primer/probes may be peptide nucleic acids in which the constituent bases are joined by peptide bonds rather than phosphodiester linkages. It will be understood by one of skill in the art that probes may bind target sequences lacking complete complementarity with the primer/probe sequence depending upon the stringency of the hybridization conditions. The primers/probes are preferably directly labeled as with isotopes, chromophores, lumiphores, chromogens, or indirectly labeled such as with biotin to which a streptavidin complex may later bind. By assaying for the presence or absence of the primer/probe, one can detect the presence or absence of the select sequence or subsequence.

As used herein, the term “expression level of nucleic acid sequences” refers to the amount of mRNA transcribed from the corresponding genes that are present in a biological sample. The expression level can be detected with or without comparison to a level from a control sample or a level expected of a control sample.

As used herein, the term “down-regulated” refers to nucleic acid molecules whose levels decrease by at least25%, or 30%, or 40% or 50% or greater in disease or cancerous cells or tissues as compared with the levels in normal, disease-free cells or tissues.

As used herein, the term “methylation” refers to the covalent attachment of a methyl group at the C5-position of the nucleotide base cytosine within the CpG dinucleotides of gene regulatory region. The term “hypermethylation” refers to the methylation state corresponding to an increased presence of 5-methyl-cytosine (“5-mCyt”) at one or a plurality of CpG dinucleotides within a DNA sequence of a test DNA sample, relative to the amount of 5-mCyt found at corresponding CpG dinucleotides within a normal control DNA sample. The term “methylation state” or “methylation status” or “methylation level” or “the degree of methylation” refers to the presence or absence of 5-mCyt at one or a plurality of CpG dinucleotides within a DNA sequence. As used herein, the terms “methylation status” or “methylation state” or “methylation level” or “degree of methylation” are used interchangeably. A methylation site refers to a sequence of contiguous linked nucleotides that is recognized and methylated by a sequence-specific methylase. Furthermore, a methylation site also refers to a specific cytosine of a CpG dinucleotide in the CpG islands. A methylase is an enzyme that methylates (i.e., covalently attaches a methyl group to) one or more nucleotides at a methylation site.

As used here, the term “CpG islands” are short DNA sequences rich in the CpG dinucleotide and defined as sequences greater than 200 bp in length, with a GC content greater than 0.5 and an observed to expected ratio based on GC content greater than 0.6. See Gardiner-Garden and Frommer, “CpG islands in vertebrate genomes,” J. Mol. Biol. 196(2): 261-282 (1987). CpG islands were associated with the 5′ ends of all housekeeping genes and many tissue-specific genes, and with the 3′ ends of some tissue-specific genes. A few genes contain both the 5′ and the 3′ CpG islands, separated by several thousand base pairs of CpG-depleted DNA. The 5′ CpG islands extended through 5′-flanking DNA, exons, and introns, whereas most of the 3′ CpG islands appeared to be associated with exons. CpG islands are generally found in the same position relative to the transcription unit of equivalent genes in different species, with some notable exceptions. CpG islands have been estimated to constitute 1%-2% of the mammalian genome, and are found in the promoters of all housekeeping genes, as well as in a less conserved position in 40% of genes showing tissue-specific expression. The persistence of CpG dinucleotides in CpG islands is largely attributed to a general lack of methylation of CpG islands, regardless of expression status. The term “CpG site” refers to the CpG dinucleotide within the CpG islands. CpG islands are typically, but not always, between about 0.2 to about 1 kb in length.

The term “significant increase in the expression levels” refers to an increase from the standard level by an amount greater than the standard error of the assay employed to assess expression. Preferably, the increase is at least about 10%, preferably at least about 15%, about 25%, about 30%, about 40%, about 50%, about 65%, about 75%, about 85%, about 90%, about 95% or greater.

The term “significant increase in the levels of the proteins” as used herein, refers to an increase in protein levels by an amount greater than the standard error of the assay employed to assess expression. Preferably, the increase is at least about 15%, preferably at least about 25%, 35%, 50%, or greater.

As used herein, the term “standard expression level of nucleic acid sequences” refers to the amount of mRNA transcribed from the corresponding genes that are present in a biological sample representative of healthy, disease-free subjects. The term “standard expression level of nucleic acid sequences” can also refer to an established level of mRNA representative of the disease-free population, that has been previously established based on measurement from healthy, disease-free subjects.

As used herein, the term “cancerous cell” or “cancer cell”, used either in the singular or plural form, refers to cells that have undergone a malignant transformation that makes them pathological to the host organism. Malignant transformation is a single- or multi-step process, which involves in part an alteration in the genetic makeup of the cell and/or the gene expression profile. Malignant transformation may occur either spontaneously, or via an event or combination of events such as drug or chemical treatment, radiation, fusion with other cells, viral infection, or activation or inactivation of particular genes. Malignant transformation may occur in vivo or in vitro, and can if necessary be experimentally induced. Malignant cells may be found within the well-defined tumor mass or may have metastasized to other physical locations. A feature of cancer cells is the tendency to grow in a manner that is uncontrollable by the host, but the pathology associated with a particular cancer cell may take any form. Primary cancer cells (that is, cells obtained from near the site of malignant transformation) can be readily distinguished from non-cancerous cells by well-established pathology techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells.

As used herein, the term “subject” refers to any human or non-human organism.

As used herein, “individual” refers to a mammal, preferably a human.

As used herein, “detecting” refers to the identification of the presence or absence of a molecule in a sample. Where the molecule to be detected is a polypeptide, the step of detecting can be performed by binding the polypeptide with an antibody that is detectably labeled. A detectable label is a molecule which is capable of generating, either independently, or in response to a stimulus, an observable signal. A detectable label can be, but is not limited to a fluorescent label, a chromogenic label, a luminescent label, or a radioactive label. Methods for “detecting” a label include quantitative and qualitative methods adapted for standard or confocal microscopy, FACS analysis, and those adapted for high throughput methods involving multi-well plates, arrays or microarrays. One of skill in the art can select appropriate filter sets and excitation energy sources for the detection of fluorescent emission from a given fluorescent polypeptide or dye. “Detecting” as used herein can also include the use of multiple antibodies to a polypeptide to be detected, wherein the multiple antibodies bind to different epitopes on the polypeptide to be detected. Antibodies used in this manner can employ two or more detectable labels, and can include, for example a FRET pair. A polypeptide molecule is “detected” according to the present invention when the level of detectable signal is at all greater than the background level of the detectable label, or where the level of measured nucleic acid is at all greater than the level measured in a control sample.

As used herein, “detecting” also refers to detecting the presence of a target nucleic acid molecule (e.g., a nucleic acid molecule encoding the marker gene) during a process wherein the signal generated by a directly or indirectly labeled probe nucleic acid molecule (capable of hybridizing to a target in a serum sample) is measured or observed. Thus, detection of the probe nucleic acid is directly indicative of the presence, and thus the detection, of a target nucleic acid, such as a sequence encoding a marker gene. For example, if the detectable label is a fluorescent label, the target nucleic acid is “detected” by observing or measuring the light emitted by the fluorescent label on the probe nucleic acid when it is excited by the appropriate wavelength, or if the detectable label is a fluorescence/quencher pair, the target nucleic acid is “detected” by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is “detected” by, for example, autoradiography. Methods and techniques for “detecting” fluorescent, radioactive, and other chemical labels may be found in Ausubel et al. (1995, Short Protocols in Molecular Biology, 3^{rd} Ed. John Wiley and Sons, Inc.). Alternatively, a nucleic acid may be “indirectly detected” wherein a moiety is attached to a probe nucleic acid which will hybridize with the target, such as an enzyme activity, allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers which are specifically designed to hybridize with a portion of the target nucleic acid sequence. Quantitative amplification methods, such as, but not limited to TaqMan, may also be used to “detect” a target nucleic acid according to the invention. A nucleic acid molecule is “detected” as used herein where the level of nucleic acid measured (such as by quantitative PCR), or the level of detectable signal provided by the detectable label is at all above the background level.

As used herein, “detecting” further refers to detecting methylation state or status on a specific CpG site of a target nucleic acid molecule that are indicative of a disease condition in a cell or tissue. The methylation state or status on a specific CpG site of a target nucleic acid molecule can provide useful information for diagnosis, disease monitoring, and therapeutic approaches. Various methods known in the art may be used for determining the methylation status of specific CpG dinucleotides. Such methods include but are not limited to, restriction landmark genomic scanning, see Kawai et al., “Comparison of DNA methylation patterns among mouse cell lines by restriction landmark genomic scanning,” Mol. Cell Biol. 14(11): 7421-7427 (1994); methylated CpG island amplification, see Toyota et al., “Identification of differentially methylated sequences in colorectal cancer by methylated CpG island amplification,” Cancer Res., 59: 2307-2312 (1999), see also WO00/26401A1; differential methylation hybridization, see Huang et al., “Methylation profiling of CpG islands in human breast cancer cells,” Hum. Mol. Genet., 8: 459-470 (1999); methylation-specific PCR (MSP), see Herman et al., “Methylation-specific PCR: a novel PCR assay for methylation status of CpG islands,” PNAS USA 93: 9821-9826 (1992), see also U.S. Pat. No. 5,786,146; methylation-sensitive single nucleotide primer extension (Ms-SnuPE), see U.S. Pat. No. 6,251,594; combined bisulfite restriction analysis (COBRA), see Xiong and Laird, “COBRA: a sensitive and quantitative DNA methylation assay,” Nucleic Acids Research, 25(12): 2532-2534 (1997); bisulfite genomic sequencing, see Frommer et al., “A genomic sequencing protocol that yields a positive display of 5-methylcytosine residues in individual DNA strands,” PNAS USA, 89: 1827-1831 (1992); and methylation-specific primer extension (MSPE), etc.

As used herein, “detecting” refers further to the early detection of disease, such as cancer, particularly colorectal cancer in a patient, wherein “early” detection refers to the detection of colorectal cancer at Dukes stage A or preferably, prior to a time when the colorectal cancer is morphologically able to be classified in a particular Dukes stage. “Detecting” as used herein further refers to the detection of colorectal cancer recurrence in an individual, using the same detection criteria as indicated above. “Detecting” as used herein still further refers to the measuring of a change in the degree of colorectal cancer before and/or after treatment with a therapeutic compound. In this case, a change in the degree of colorectal cancer in response to a therapeutic compound refers to an increase or decrease in the expression of the marker genes including one or more colorectal cancer associated markers, or alternatively, in the amount of the marker gene polypeptide including one or more colorectal cancer associated markers presented in a clinical sample by at least 10% in response to the presence of a therapeutic compound relative to the expression level in the absence of the therapeutic compound. In addition, a change in the degree of colorectal cancer in response to a therapeutic compound also refers to a change in methylation of colorectal cancer associated markers.

II Identification of the Down-Regulated Nucleic Acid Marker Sequences in Disease Cells

In one aspect, the present invention pertains to identification of down-regulated (under-expressed) nucleic acid marker sequences in a biological sample from a patient having or suspected of a disease or disorder, such as cancer or a pre-malignant condition. In general, the method of identifying the nucleic acid marker sequences includes (1) providing a pool of target nucleic acids preferably derived from both disease and normal cells and/or tissues and preferably comprising RNA transcripts of the target nucleic acid marker sequences or nucleic acids derived from the RNA transcripts; (2) hybridizing the nucleic acid samples to one or more probes; and (3) detecting the hybridized nucleic acids and determining the expression levels derived from the diseased cells/tissues relative to the expression levels of the same nucleic acids from normal cells and/or tissues. Various conventional methods known in the art may be employed to identify the nucleic acid marker sequences that are down-regulated in a disease, especially cancer. In one embodiment, microarrays such as DNA arrays are employed in the method.

The nucleic acids can be isolated/extracted from any source. Preferably, the sample may be obtained from cell lines, blood, sputum, stool, urine, serum, cerebro-spinal fluid, tissue embedded in paraffin, for example, tissue from eyes, intestine, kidneys, brain, heart, prostate, lungs, breast or liver, histological slides, and all possible combinations thereof.

A variety of methods have been employed to achieve this end. They include differential screening of cDNA libraries with selective probes, subtractive hybridization utilizing DNA/DNA hybrids or DNA/RNA hybrids, RNA fingerprinting and differential display (Mather, et al. (1981) Cell 23:369-378; Hedrick et al. (1984) Nature 308:149-153; Davis et al. (1992) Cell 51:987-1000; Welsh et al. (1992) Nucleic Acids Res. 20:4965-4970; and Liang and Pardee (1992) Science 257:967-971). Recently, PCR-coupled subtractive processes have also been reported (Straus and Ausubel (1990) Proc. Natl. Sci. USA 87:1889-1893; Sive and John (1988) Nucleic Acids Res. 16:10937; Wieland et al. (1990) Proc. Natl. Acad. Sci. USA 87:2720-2724; Wang and Brown (1991) Proc. Natl. Acad. Sci. USA 88:11505-11509; Lisitsyn et al. (1993) Science 259:946-951; Zeng et al. (1994) Nucleic Acids Res. 22:4381-4385; Hubank and Schatz (1994) Nucleic Acids Res. 22:5640-5648). Also recently, a microarray technology (DNA chips) developed by Affymetrix (Santa Clara, Calif.) has been used as a powerful tool to simultaneously identify a large number of differentially expressed nucleic acid marker sequences in a biological sample. Each of these methods can be employed in the present invention and is hereby incorporated by reference in their entirety.

By using the Affymetrix chips (GeneChip Human Genome U133 Set), the inventors of the present invention identified the down-regulated nucleic acid marker sequences that have shown at least about two-fold decrease in expression levels in biological samples from disease cells and/or tissue, including colon cancer-derived cells and/or tissue, relative to the expression level in samples from normal cells and/or tissue, e.g., normal colon tissue and/or normal non-colon tissue. Table 1 describes the identified nucleic acid marker sequences that are down-regulated in tumor cells and/or tissue, e.g., colon cancer-derived cells and/or tissue. The sequences dictated by SEQ ID NO's are genomic sequences of the corresponding genes.

TABLE 1
Sequences with expression down-regulated in CRC as compared to normal tissues

| | | | Cancer | | Normal | | |
|---|---|---|---|---|---|---|---|
| Gene name | GenBank ID | Unigene ID | Mean | Median | Mean | Median | SEQ ID NO |
| CLDN8 | AL049977.1 | Hs.162209 | 0.5 | 0.4 | 24.7 | 16.0 | 1 |
| CLCA4 | NM_012128.2 | Hs.227059 | 0.1 | 0.1 | 12.8 | 13.8 | 2 |
| AQP8 | NM_001169.1 | Hs.176658 | 0.3 | 0.3 | 18.2 | 12.8 | 3 |
| MS4A12 | NM_017716.1 | Hs.272789 | 0.1 | 0.0 | 13.5 | 12.7 | 4 |
| LOC339479 | BF589529 | Hs.106642 | 0.7 | 0.5 | 13.4 | 12.1 | 5 |
| GUCA2B | NM_007102.1 | Hs.32966 | 0.0 | 0.0 | 10.3 | 10.9 | 6 |
| GCG | NM_002054.1 | Hs.1460 | 0.1 | 0.1 | 17.0 | 9.6 | 7 |
| CA1 | NM_001738.1 | Hs.23118 | 0.1 | 0.1 | 8.4 | 9.5 | 8 |
| PYY | NM_004160.1 | Hs.169249 | 0.2 | 0.1 | 12.4 | 9.2 | 9 |
| UGT2B15 | NM_001076.1 | Hs.150207 | 0.4 | 0.3 | 9.2 | 8.5 | 10 |
| GUCA1B | NM_002098.1 | Hs.284258 | 0.1 | 0.1 | 7.4 | 7.7 | 11 |
| | AW519168 | Hs.293441 | 0.6 | 0.6 | 8.8 | 7.6 | 12 |
| UGT2B17 | NM_001077.1 | Hs.183596 | 0.3 | 0.1 | 7.2 | 6.9 | 13 |
| CEACAM7 | L31792.1 | Hs.74466 | 0.3 | 0.1 | 6.6 | 6.5 | 14 |
| CEACAM7 | AF006623.1 | Hs.74466 | 0.3 | 0.2 | 6.5 | 6.4 | 14 |
| TU3A | AL050264.1 | Hs.8022 | 0.5 | 0.3 | 5.9 | 5.4 | 15 |
| SPINK5 | NM_006846.1 | Hs.331555 | 0.3 | 0.2 | 6.8 | 5.3 | 16 |
| NR1H4 | NM_005123.1 | Hs.171683 | 0.7 | 0.7 | 5.0 | 5.3 | 17 |
| TNFRSF17 | NM_001192.1 | Hs.2556 | 0.5 | 0.1 | 5.1 | 5.0 | 18 |
| CLCA1 | AF127036.1 | Hs.194659 | 0.3 | 0.1 | 4.8 | 4.5 | 19 |
| PYY | D13902.1 | Hs.169249 | 0.3 | 0.2 | 5.1 | 4.3 | 9 |
| | AV733266 | Hs.76325 | 0.5 | 0.3 | 3.9 | 4.3 | 20 |
| ANPEP | NM_001150.1 | Hs.1239 | 0.4 | 0.4 | 7.2 | 4.1 | 21 |
| SLC26A2 | AI025519 | Hs.29981 | 0.4 | 0.1 | 4.0 | 4.1 | 22 |
| MT1K | R06655 | Hs.188518 | 0.4 | 0.1 | 5.0 | 4.0 | 23 |
| MMP28 | NM_024302.1 | Hs.231958 | 0.4 | 0.3 | 4.1 | 3.7 | 24 |
| ADAMDEC1 | NM_014479.1 | Hs.145296 | 0.6 | 0.4 | 3.5 | 3.7 | 25 |
| RNAHP | AF078844.1 | Hs.8765 | 0.7 | 0.6 | 3.7 | 3.6 | 26 |
| FLJ21511 | NM_025087.1 | Hs.288462 | 0.2 | 0.2 | 3.7 | 3.6 | 27 |
| ATOH1 | NM_005172.1 | Hs.247685 | 0.5 | 0.2 | 3.7 | 3.6 | 28 |
| | AI732905 | Hs.184507 | 0.0 | 0.0 | 3.6 | 3.6 | 29 |
| | S55735.1 | Hs.293441 | 0.3 | 0.2 | 3.4 | 3.6 | 30 |
| ADH1C | NM_000669.2 | Hs.2523 | 0.1 | 0.1 | 4.0 | 3.5 | 31 |
| | M21692.1 | | 0.6 | 0.4 | 3.2 | 3.5 | 32 |
| PDE9A | NM_002606.1 | Hs.18953 | 0.2 | 0.1 | 4.0 | 3.4 | 33 |
| SLC4A4 | AF011390.1 | Hs.5462 | 0.3 | 0.3 | 3.8 | 3.4 | 34 |
| RNAHP | BF246115 | Hs.8765 | 0.6 | 0.4 | 3.4 | 3.4 | 26 |
| TNA | NM_003278.1 | Hs.65424 | 0.7 | 0.6 | 3.7 | 3.3 | 35 |
| CA4 | NM_000717.2 | Hs.89485 | 0.2 | 0.1 | 3.6 | 3.3 | 36 |
| PRV1 | NM_020406.1 | Hs.232165 | 0.3 | 0.3 | 4.2 | 3.2 | 37 |
| FLJ20132 | NM_017682.1 | Hs.190222 | 0.5 | 0.5 | 3.7 | 3.1 | 38 |
| FLJ21458 | NM_024850.1 | Hs.189109 | 0.6 | 0.4 | 3.6 | 3.1 | 39 |
| LGALS2 | NM_006498.1 | Hs.113987 | 0.3 | 0.2 | 3.1 | 3.1 | 40 |
| EDN3 | NM_000114.1 | Hs.1408 | 0.5 | 0.5 | 3.0 | 3.1 | 41 |
| HSD3B2 | NM_000198.1 | Hs.825 | 0.6 | 0.3 | 7.3 | 3.0 | 42 |
| CA4 | NM_000717.2 | Hs.89485 | 0.1 | 0.0 | 3.8 | 3.0 | 36 |
| | AK025044.1 | | 0.3 | 0.1 | 3.5 | 3.0 | 43 |
| FLJ21511 | NM_025087.1 | Hs.288462 | 0.1 | 0.0 | 2.9 | 3.0 | 27 |
| SGK | NM_005627.1 | Hs.296323 | 0.6 | 0.5 | 3.0 | 2.8 | 44 |
| HPGD | U63296.1 | Hs.77348 | 0.7 | 0.6 | 2.9 | 2.8 | 45 |
| KIAA0523 | BF115148 | Hs.16032 | 0.4 | 0.3 | 2.9 | 2.8 | 46 |
| BCAS1 | NM_003657.1 | Hs.129057 | 0.5 | 0.4 | 3.0 | 2.7 | 47 |
| UGT1A8 | NM_019076.1 | Hs.278741 | 0.4 | 0.3 | 2.7 | 2.7 | 48 |
| MT1F | M10943 | | 0.5 | 0.3 | 2.6 | 2.7 | 49 |
| FMO5 | AK022172.1 | Hs.14286 | 0.6 | 0.5 | 2.5 | 2.7 | 50 |
| SCGB2A1 | NM_002407.1 | Hs.97644 | 0.3 | 0.1 | 4.0 | 2.6 | 51 |
| ABCA8 | NM_007168.1 | Hs.38095 | 0.5 | 0.4 | 3.6 | 2.6 | 52 |
| FLJ32987 | NM_016459.1 | Hs.122492 | 0.4 | 0.2 | 3.6 | 2.6 | 53 |
| RDHL | NM_005771.1 | Hs.179608 | 0.2 | 0.1 | 3.2 | 2.6 | 54 |
| FLJ22595 | NM_025047.1 | Hs.287702 | 0.3 | 0.3 | 3.1 | 2.6 | 55 |
| CHGA | NM_001275.2 | Hs.172216 | 0.2 | 0.1 | 3.5 | 2.5 | 56 |
| LOC63928 | NM_022097.1 | Hs.178589 | 0.2 | 0.0 | 2.8 | 2.5 | 57 |
| SCNN1B | NM_000336.1 | Hs.37129 | 0.3 | 0.3 | 2.7 | 2.5 | 58 |
| ADH1B | M24317.1 | Hs.4 | 0.7 | 0.5 | 2.7 | 2.5 | 59 |
| MT1H | NM_005951.1 | Hs.2667 | 0.6 | 0.4 | 2.4 | 2.5 | 60 |
| SST | NM_001048.1 | Hs.12409 | 0.6 | 0.5 | 4.9 | 2.4 | 61 |
| FLJ12768 | NM_025163.1 | Hs.289077 | 0.6 | 0.5 | 2.7 | 2.4 | 62 |
| MT1G | NM_005950.1 | Hs.334409 | 0.6 | 0.5 | 2.5 | 2.4 | 63 |
| GPR2 | NM_016602.1 | Hs.278446 | 0.7 | 0.7 | 2.4 | 2.4 | 64 |
| GLUC | NM_020973.1 | Hs.146182 | 0.4 | 0.4 | 3.5 | 2.3 | 65 |
| ABCG2 | AF098951.2 | Hs.194720 | 0.5 | 0.4 | 2.8 | 2.3 | 66 |
| HPGD | NM_000860.1 | Hs.77348 | 0.6 | 0.4 | 2.6 | 2.3 | 45 |
| GPT | NM_005309.1 | Hs.103502 | 0.4 | 0.2 | 2.5 | 2.3 | 67 |
| CEACAM1 | X16354.1 | Hs.50964 | 0.6 | 0.6 | 2.5 | 2.3 | 68 |
| CEACAM1 | NM_001712.1 | Hs.50964 | 0.6 | 0.5 | 3.0 | 2.2 | 68 |
| VIP | NM_003381.1 | Hs.53973 | 0.7 | 0.5 | 3.0 | 2.2 | 69 |
| NEDD4L | AB007899.1 | Hs.12017 | 0.6 | 0.5 | 2.7 | 2.2 | 70 |
| NPY1R | NM_000909.1 | Hs.169266 | 0.6 | 0.4 | 2.6 | 2.2 | 71 |
| CEACAM1 | D12502.1 | Hs.50964 | 0.6 | 0.5 | 2.6 | 2.2 | 68 |
| MGC12335 | AL022724 | | 0.6 | 0.5 | 2.5 | 2.2 | 72 |
| IGLJ3 | D01059.1 | Hs.181125 | 0.7 | 0.6 | 2.3 | 2.2 | 73 |
| MUC2 | NM_002457.1 | Hs.315 | 0.4 | 0.1 | 2.2 | 2.2 | 74 |
| TNXB | M25813.1 | Hs.169886 | 0.5 | 0.4 | 2.6 | 2.1 | 75 |
| DKFZp547M236 | NM_018713.1 | Hs.20981 | 0.4 | 0.3 | 2.6 | 2.1 | 76 |
| HPGD | J05594.1 | Hs.77348 | 0.5 | 0.4 | 2.5 | 2.1 | 45 |
| FLJ10718 | NM_018192.1 | Hs.42824 | 0.5 | 0.3 | 2.5 | 2.1 | 77 |
| HSD17B2 | NM_002153.1 | Hs.155109 | 0.6 | 0.3 | 2.3 | 2.1 | 78 |
| CACNB2 | AI040163 | Hs.30941 | 0.6 | 0.4 | 2.3 | 2.1 | 79 |
| | NM_007116.1 | | 0.6 | 0.5 | 2.8 | 2.0 | 80 |
| MUCDHL | NM_021924.1 | Hs.165619 | 0.4 | 0.3 | 2.5 | 2.0 | 81 |
| HRASLS2 | NM_017878.1 | Hs.272805 | 0.8 | 0.8 | 2.3 | 2.0 | 82 |
| IL1R2 | NM_004633.1 | Hs.25333 | 0.3 | 0.3 | 2.2 | 2.0 | 83 |
| CYP2C18 | NM_000772.1 | Hs.702 | 0.8 | 0.6 | 2.5 | 1.9 | 84 |
| TNXB | BE044614 | Hs.169886 | 0.5 | 0.4 | 2.5 | 1.9 | 75 |
| ENTPD5 | NM_001249.1 | Hs.80975 | 0.5 | 0.5 | 2.3 | 1.9 | 85 |
| FLJ10970 | NM_018286.1 | Hs.173233 | 0.5 | 0.5 | 2.3 | 1.9 | 86 |
| CLDN5 | NM_003277.1 | Hs.110903 | 0.7 | 0.7 | 2.1 | 1.9 | 87 |
| GPR105 | NM_014879.1 | Hs.2465 | 0.7 | 0.6 | 2.0 | 1.9 | 88 |
| | AB002438.1 | | 0.7 | 0.7 | 3.1 | 1.8 | 89 |
| SPINK4 | NM_014471.1 | Hs.129778 | 0.5 | 0.2 | 2.7 | 1.8 | 90 |
| FHL1 | AF098518.1 | Hs.239069 | 0.9 | 0.7 | 2.5 | 1.8 | 91 |
| FHL1 | AF220153.1 | Hs.239069 | 0.7 | 0.6 | 2.1 | 1.8 | 91 |
| SI | NM_001041.1 | Hs.2996 | 0.4 | 0.1 | 1.9 | 1.8 | 92 |
| DEFB1 | U73945.1 | Hs.32949 | 0.7 | 0.4 | 2.3 | 1.7 | 93 |
| KLRB1 | NM_002258.1 | Hs.169824 | 0.7 | 0.6 | 2.2 | 1.7 | 94 |
| POU2AF1 | NM_006235.1 | Hs.2407 | 0.5 | 0.3 | 2.0 | 1.7 | 95 |
| MEP1B | NM_005925.1 | Hs.194777 | 0.7 | 0.6 | 2.8 | 1.6 | 96 |
| FHL1 | U29538.1 | Hs.239069 | 0.9 | 0.8 | 2.2 | 1.6 | 91 |
| TRG | M16768.1 | Hs.112259 | 0.7 | 0.6 | 2.1 | 1.6 | 97 |
| EMP1 | NM_001423.1 | Hs.79368 | 0.8 | 0.7 | 2.0 | 1.6 | 98 |
| DNASE1L3 | NM_004944.1 | Hs.88646 | 0.6 | 0.5 | 2.0 | 1.6 | 99 |
| PDK4 | NM_002612.1 | Hs.299221 | 0.7 | 0.6 | 2.4 | 1.5 | 100 |
| EMP1 | NM_001423.1 | Hs.79368 | 0.7 | 0.6 | 2.2 | 1.5 | 98 |
| SLC20A1 | NM_005415.2 | Hs.78452 | 0.8 | 0.7 | 2.0 | 1.5 | 101 |
| MMP15 | NM_002428.1 | Hs.80343 | 0.6 | 0.4 | 2.0 | 1.5 | 102 |
| BCHE | NM_000055.1 | Hs.1327 | 0.7 | 0.8 | 1.9 | 1.5 | 103 |
| | AK023795.1 | | 0.7 | 0.7 | 1.9 | 1.5 | 104 |
| | AL137750.1 | | 0.8 | 0.7 | 3.5 | 1.4 | 105 |
| C7 | NM_000587.1 | Hs.78065 | 0.7 | 0.4 | 1.9 | 1.3 | 106 |
| MYH11 | NM_022870.1 | Hs.78344 | 0.8 | 0.8 | 1.7 | 1.3 | 107 |
| FLJ20225 | NM_019062.1 | Hs.124835 | 0.6 | 0.6 | 1.5 | 1.3 | 108 |
| CA2 | M36532.1 | Hs.155097 | 0.1 | 0.0 | 3.0 | 3.1 | 109 |
| SLC4A4 | NM_003759.1 | Hs.5462 | 0.1 | 0.1 | 3.0 | 2.9 | 34 |
| FCGBP | NM_003890.1 | Hs.111732 | 0.1 | 0.0 | 2.4 | 2.2 | 110 |
| CEACAM7 | NM_006890.1 | Hs.74466 | 0.2 | 0.1 | 3.0 | 3.0 | 14 |
| HMGCS2 | NM_005518.1 | Hs.59889 | 0.3 | 0.2 | 2.5 | 2.2 | 111 |
| PLAC8 | NM_016619.1 | Hs.107139 | 0.3 | 0.1 | 1.9 | 1.8 | 112 |
| FLJ22543 | NM_024308.1 | Hs.8949 | 0.4 | 0.3 | 2.5 | 2.3 | 113 |
| | NM_017678.1 | Hs.179100 | 0.3 | 0.0 | 2.1 | 1.8 | 114 |
| PCK1 | NM_002591.1 | Hs.1872 | 0.4 | 0.4 | 2.6 | 2.9 | 115 |
| KRT20 | AI732381 | Hs.84905 | 0.4 | 0.4 | 2.3 | 2.2 | 116 |
| PIGR | NM_002644.1 | Hs.205126 | 0.4 | 0.1 | 1.7 | 1.7 | 117 |
| EKI1 | NM_018638.2 | Hs.120439 | 0.8 | 0.8 | 3.6 | 1.5 | 118 |
| HIG1 | BE739519 | Hs.7917 | 0.4 | 0.4 | 1.7 | 1.6 | 119 |
| | AF333388.1 | | 0.6 | 0.3 | 2.1 | 2.3 | 120 |
| | AL031602 | | 0.5 | 0.4 | 2.0 | 2.1 | 121 |
| CKBB | NM_001823.1 | Hs.173724 | 0.6 | 0.5 | 2.1 | 2.0 | 122 |
| CES2 | BF033242 | Hs.282975 | 0.5 | 0.4 | 1.8 | 1.9 | 123 |
| | NM_022129.1 | Hs.16341 | 0.6 | 0.4 | 1.9 | 1.9 | 124 |
| MT1X | NM_005952.1 | Hs.374950 | 0.6 | 0.5 | 1.9 | 2.0 | 125 |
| MT2A | NM_005953.1 | Hs.118786 | 0.7 | 0.5 | 1.8 | 1.6 | 126 |
| FHL1 | NM_001449.1 | Hs.239069 | 0.6 | 0.6 | 1.9 | 1.8 | 91 |
| STK39 | NM_002450.1 | Hs.199263 | 0.7 | 0.6 | 1.8 | 1.9 | 127 |
| SFN | X57348 | Hs.184510 | 0.7 | 0.6 | 1.5 | 1.2 | 128 |
| GPX3 | NM_002084.2 | Hs.386793 | 0.8 | 0.7 | 1.4 | 1.3 | 129 |

Accordingly, the present invention further provides nucleic acid marker sequences in Table 1 that are under-expressed (down-regulated) by at least about 2 fold, at least about 5 fold, at least about 10 fold, at least about 20 fold, or at least about 50 fold. In one embodiment, the present invention encompasses nucleic acid marker sequences that are under-expressed (down-regulated) in disease cells and/or tissue, especially in colon cancer cells and/or tissue and/or colon cancer-derived cell lines. In a preferred embodiment, the nucleic acid marker sequences are under-expressed (down-regulated) by at least about 2 fold, at least about 5 fold, at least about 10 fold, at least about 20 fold, or at least about 50 fold.

The present invention also encompasses nucleic acid sequences which differ from the nucleic acid marker sequences identified in Tables 1 and 2, but which produce the same phenotypic effect, for example, an allelic or splice variant.

The present invention further encompasses polynucleotides which are at least 85%, or at least 90%, or more preferably equal to or greater than 95% identical to the sequences of the RNA transcripts or cDNAs of the nucleic acid marker sequences. Sequence identity as used herein refers to the proportion of base matches between two nucleic acid sequences or the proportion amino acid matches between two amino acid sequences. When sequence homology is expressed as a percentage, e.g., 50%, the percentage denotes the proportion of matches over the length of sequence from one sequence that is compared to some other sequence.

III Identification of CpG Islands

In another aspect, the present invention pertains to the identification of CpG islands on the down-regulated marker sequences including but not limited to, the marker sequences described in Table 1. In selecting a CpG island, the identification preferably uses the Gardiner-Garden and Frommer definition for CpG islands. See Gardiner-Garden and Frommer, “CpG islands in vertebrate genomes,” J. Mol. Biol. 196(2): 261-282 (1987). That is, a CpG island must have sequences greater than 200 bp in length, with a GC content greater than 0.5 and an observed to expected ratio based on GC content greater than 0.6. Moreover, the sequences that span from about 1000 bp upstream of the start of the first exon to about 1000 bp downstream of the first exon are searched for the presence of any CpG island. The search for CpG islands can be made manually or with programs. For example Takai and Jones has developed a web program for searching CpG islands, which is incorporated by reference in its entirety herein. See Takai and Jones, “The CpG Island Searcher: A New WWW Resource,” In Silico Biol. Feb. 4, 2003. See also the web program entitled “CpG Island Searcher” designed by Takai, Daiya, or Takai, D and Jones, P., “Comphrensive analysi of CpG islands in human chromosomes 21 and 22,” PNSA USA, 99(6): 3740-3745. See also a web program entitled “CpGPlot/CpGReport/Isochore,” made by EMBL-EBI European Bioinformatics Institute, or Rice, P et al., “EMBOSS: the European Molecular Biology Open Software Suite,” Trends Genet, 16(6):276-7 (2000), or Gardiner-Garden, M and Frommer, M, “CpG islands in vertebrate genomes,” J. Mol. Biol., 196(2):261-82 (1987), or Bernardi, G, “Isochores and the evolutionary genomics of vertebrates,” Gene, 241(1): 3-17 (2000), or Pesole, G. et al., “Isochore specificity of AUG initiator context of human genes,” FEBS Lett., 464(1-2): 60-62 (1999), or Larsen, F. et al., “CpG islands as gene markers in the human genome,” Genomics, 13(4): 1095-1107 (1992). Based on a CpG-island-extraction algorithm, the web program determines the location of CpG islands using parameters (lower limit of % GC, observed CpG/expected CpG ratio, and length) set by the user, to display the value of parameters on each CpG island, and provide a graphical map of CpG dinucleotide distribution and borders of CpG islands. A command-line version of the web program can also be used to search larger sequences.

For some genes, the genomic sequences are available and the promoter regions have been identified, thereby, it is relatively easy for one to identify a potential CpG island within the promoter-first exon regions. For other genes, the promoter regions of genomic sequences are not yet identified. Therefore, in one embodiment, the present invention provides a method of identifying CpG islands when the promoter regions of genomic sequences are not yet identified. Such method includes, for example, first identifying the transcription start site, then analyzing the CpG islands in the promoter regions. For example, Suzuki et al. describe an “oligo-capping” method to identify and characterize the promoter regions and CpG islands across the promoter regions of human genes. See Suzuki, Y. et al., “Identification and Characterization of the Potential Promoter Regions of 1031 Kinds of Human Genes,” Genome Research, 677-684 (2001), which is incorporated by reference herein. In this method, the promoters of genes are first identified by the oligo-capped method. See Suzuki, et al., “Statical analysis of the 5′ untranslated region of human mRNA using oligo-capped cDNA libraries,” Genomics, 64: 286-297 (2000). The mRNA start sites are then mapped onto the genomic sequences with the help of BLASTN program and CLUSTASLW program. For each gene, the genomic sequences between 1000 bp upstream and 1000 bp downstream are retrieved as regions for identification of CpG islands. The promoter regions are defined as the sequences extending from about 1000 bp, preferably about 500 bp upstream to about 1000 bp, preferably 500 bp downstream of the identified mRNA start sites. For analysis of CpG islands, the moving average for % (G+C) and the CpG ratio are calculated for each sequence, using a selected size, preferably 100 bp window moving along the sequence at 1 bp intervals. The CpG ratio is calculated according to the Gardiner-Garden and Frommer criteria: (number of CG×N)/(number of C×number of G), where N is the total number of nucleotides in the sequence being analyzed.

By applying the Gardiner-Garden and Frommer criteria and using one of the methods described above, the representative numbers of the CpG islands were identified and listed in Table 2. The sequences dictated by SEQ ID NO's are the same as the sequences designated in the column “Search parameter.”

TABLE 2
Subset of sequences containing at least one CpG island in the promoter-first exon region.

| | | | # CpG | | SEQ ID |
|---|---|---|---|---|---|
| Gene name | GenBank ID | Unigene ID | islands | Search parameter | NO |
| PYY | NM_004160.1 | Hs.169249 | 2 | 1000-exon1 + 1000 | 130 |
| ANPEP | NM_001150.1 | Hs.1239 | 1 | 1000-exon1 + 1000 | 131 |
| SLC26A2.a | AI025519 | Hs.29981 | 3 | 1000-exon1 + 1000 | 132 |
| MT1K | R06655 | Hs.188518 | 1 | 1000-exon1 + 500 | 133 |
| MMP28 | NM_024302.1 | Hs.231958 | 2 | 1000-exon1 + 500 | 134 |
| FLJ21511 | NM_025087.1 | Hs.288462 | 1 | 1000-exon1 + 500 | 135 |
| ATOH1 | NM_005172.1 | Hs.247685 | 3 | 1000-exon1 + 500 | 136 |
| PDE9A | NM_002606.1 | Hs.18953 | 3 | 1000-exon1 + 500 | 137 |
| CA4 | NM_000717.2 | Hs.89485 | 1 | 1000-exon1 + 500 | 138 |
| EDN3 | NM_000114.1 | Hs.1408 | 1 | 1000-exon1 + 500 | 139 |
| SGK | NM_005627.1 | Hs.296323 | 8 | 1000-exon 1-4 + 500 | 140 |
| HPGD | U63296.1 | Hs.77348 | 1 | 1000-exon1 + 500 | 141 |
| KIAA0523 | BF115148 | Hs.16032 | 1 | 1000-exon1 + 500 | 142 |
| MT1F | M10943 | | 1 | 1000-exon1 + 500 | 143 |
| CHGA | NM_001275.2 | Hs.172216 | 1 | 1000-exon1 + 500 | 144 |
| LOC63928 | NM_022097.1 | Hs.178589 | 1 | 1000-exon1 + 500 | 145 |
| SCNN1B | NM_000336.1 | Hs.37129 | 1 | 1000-exon1 + 500 | 146 |
| SST | NM_001048.1 | Hs.12409 | 1 | 1000-exon1 + 500 | 147 |
| FLJ12768 | NM_025163.1 | Hs.289077 | 1 | 1000-exon1 + 500 | 148 |
| MT1G | NM_005950.1 | Hs.334409 | 1 | 1000-exon1 + 500 | 149 |
| GPR2 | NM_016602.1 | Hs.278446 | 1 | 1000-exon1 + 500 | 150 |
| SLC4A4 | AF011390.1 | Hs.5462 | 2 | 1000-exon1 + 500 | 151 |
| ABCG2 | AF098951.2 | Hs.194720 | 1 | 1000-exon1 + 500 | 152 |
| | NM_015277 | | 1 | 1000-exon1 + 500 | 153 |
| NPY1R | NM_000909.1 | Hs.169266 | 1 | 1000-exon1 + 1000 | 154 |
| FLJ10718 | NM_018192.1 | Hs.42824 | 1 | 1000-exon1 + 500 | 155 |
| CACNB2 | AI040163 | Hs.30941 | 1 | 1000-exon1 + 500 | 156 |
| | BC020966 | | 1 | 1000-exon1 + 500 | 157 |
| CLDN5 | NM_003277.1 | Hs.110903 | 2 | 1000-exon1 + 500 | 158 |
| | NM_001449 | | 1 | 1000-exon1 + 500 | 159 |
| PDK4 | NM_002612.1 | Hs.299221 | 1 | 1000-exon1 + 500 | 160 |
| SLC20A1 | NM_005415.2 | Hs.78452 | 1 | 1000-exon1 + 1000 | 161 |
| MMP15 | NM_002428.1 | Hs.80343 | 1 | 1000-exon1 + 500 | 162 |
| | AK023795.1 | | 2 | 1000-exon1 + 500 | 163 |
| | AL137750.1 | | 1 | 1000-exon1 + 500 | 164 |
| CA2 | M36532.1 | Hs.155097 | 1 | 1000-exon1 + 500 | 165 |
| FCGBP | NM_003890.1 | Hs.111732 | 6 | entire genomic seq | 166 |
| PLAC8 | NM_016619.1 | Hs.107139 | 1 | 1000-exon1 + 500 | 167 |
| FLJ22543 | NM_024308.1 | Hs.8949 | 1 | 1000-exon1 + 500 | 168 |
| EKI1 | NM_018638.2 | Hs.120439 | 1 | 1000-exon1 + 500 | 169 |
| HIG1 | BE739519 | Hs.7917 | 1 | 1000-exon1 + 500 | 170 |
| | AL031602 | | 2 | 1000-exon1 + 500 | 171 |
| CES2 | BF033242 | Hs.282975 | 1 | 1000-exon1 + 500 | 172 |
| MT1X | NM_005952.1 | Hs.374950 | 1 | 1000-exon1 + 500 | 173 |
| MT2A | NM_005953.1 | Hs.118786 | 1 | 1000-exon1 + 500 | 174 |
| FHL1 | NM_001449.1 | Hs.239069 | 1 | 1000-exon1 + 500 | 175 |
| STK39 | NM_002450.1 | Hs.199263 | 1 | 1000-exon1 + 500 | 176 |
| SFN | X57348 | Hs.184510 | 1 | 1000-exon1 + 500 | 177 |
| GPX3 | NM_002084.2 | Hs.386793 | 1 | 1000-exon1 + 500 | 178 |

Accordingly, the present invention further provides CpG islands within the promoter-first exon region of genes that are down-regulated in disease including cancer cells. Once the CpG islands are identified, they can be used for a number of different techniques. In one technique, they are tested to identify sequences which are differentially methylated between maternal and paternal chromosomes. In another technique, they are tested to identify sequences which are differentially methylated between hydatidiform moles and teratomas. In another technique, they are tested to identify sequences which are differentially methylated between disease cells or tissues and normal healthy cells or tissues. In another technique, they are mapped to a genomic region. The CpG islands can be used to identify an imprinted gene adjacent to the methylated CpG island, as methylated CpG islands are markers for such genes. If a CpG island is found to map to the same region as a disease which is preferentially transmitted by one parent, an imprinted gene in the region can be identified as a candidate gene involved in transmitting the disease. The CpG islands can be used to screen populations of individuals for methylation. A sequence which is differentially methylated between individuals is a methylation polymorphism which can be used to identify individuals.

IV Verification of Methylation

In another aspect, the present invention pertains to determining whether the candidate CpG sites within the CpG islands of the down-regulated marker sequences are methylated in diseased cells or tissues. This can be performed by using methylation assays capable of determining differential methylation levels within CpG sites between diseased cells or tissues and normal cells or tissues.

Various methods may be used for determining the methylation status of specific CpG dinucleotides. Such methods include but not limited to, restriction landmark genomic scanning, see Kawai et al., “Comparison of DNA methylation patterns among mouse cell lines by restriction landmark genomic scanning,” Mol. Cell Biol. 14(11): 7421-7427 (1994); methylated CpG island amplification, see Toyota et al., “Identification of differentially methylated sequences in colorectal cancer by methylated CpG island amplification,” Cancer Res., 59: 2307-2312 (1999), see also WO00/26401A1; differential methylation hybridization, see Huang et al., “Methylation profiling of CpG islands in human breast cancer cells,” Hum. Mol. Genet., 8: 459-470 (1999); methylation-specific PCR (MSP), see Herman et al., “Methylation-specific PCR: a novel PCR assay for methylation status of CpG islands,” PNAS USA 93: 9821-9826 (1992), see also U.S. Pat. No. 5,786,146; methylation-sensitive single nucleotide primer extension (Ms-SNuPE), see U.S. Pat. No. 6,251,594; combined bisulfite restriction analysis (COBRA), see Xiong and Laird, “COBRA: a sensitive and quantitative DNA methylation assay,” Nucleic Acids Research, 25(12): 2532-2534 (1997); bisulfite genomic sequencing, see Frommer et al., “A genomic sequencing protocol that yields a positive display of 5-methylcytosine residues in individual DNA strands,” PNAS USA, 89: 1827-1831 (1992); and methylation-specific primer extension (MSPE), etc. All these methods for determining methylation status of CpG islands are incorporated by reference herein.

These methods may be roughly characterized as belonging to one of the two general categories: namely, restriction enzyme based technologies, or unmethylated cytosine conversion based technologies. The restriction enzyme based technologies use the methylation sensitive restriction endonucleases for the differentiation between methylated and unmethylated cytosines. In particular, the methylation sensitive restriction enzymes either cleave, or fail to cleave DNA according to the cytosine methylation state present in the recognition motif (e.g., the CpG sequences thereof). The digested DNA fragments are typically separated on the basis of size, and the methylation status of the sequence is thereby deduced, based on the presence or absence of particular fragments. Preferably, a post-digest PCR amplification step is added wherein a set of two oligonucleotide primers, one on each side of the methylation sensitive restriction site, is used to amplify the digested DNA. PCR products are not detectable where digestion of the subtended methylation sensitive restriction enzyme site occurs.

Cytosine conversion based technologies comprises methylation status-dependent chemical modification of CpG sequences within isolated nucleic acids, or within fragments thereof, and followed by nucleic acid analysis. Chemical reagents that are able to distinguish between methylated and non-methylated CpG dinucleotide sequences include hydrazine, which cleaves the nucleic acid, and the more preferred bisulfite treatment. Bisulfite treatment followed by alkaline hydrolysis specifically converts non-methylated cytosine to uracil, leaving 5-methylcytosine unmodified. See Olek A. et al., “A modified and improved method for bisulfite based cytosine methylation analysis,” Nucleic Acids Res., 24:5064-5066 (1996). The bisulfite-treated DNA may then be analyzed by conventional molecular biology techniques, such as PCR amplification, sequencing, and detection comprising oligonucleotide hybridization.

In one preferred embodiment, the MSP method is employed in the present invention. In this method, the DNA of interest is treated such that methylated and non-methylated cytosines are differentially modified (e.g., by bisulfite treatment) in a manner discernable by their hybridization behavior. PCR primers specific to each of the methylated and non-methylated states of the DNA are used in PCR amplification. Products of the amplification reaction are then detected, allowing for the deduction of the methylation status of the CpG position within the genomic DNA.

In another preferred embodiment, the bisulfite genomic sequencing method is employed. In this method, nucleic acids, preferably genomic DNAs are treated with bisulfite, followed by PCR amplification of the bisulfite treated nucleic acids and sequencing of the amplified nucleic acids.

In yet another preferred embodiment, the MSPE method is employed. This method includes chemically modifying the CpG sites, converting the non-methylated cytosines into uracil, leaving the 5′-methylated cytosine unmodified. The chemically treated nucleic acids such as DNA may then be amplified by conventional molecular biology techniques including PCR amplification. The methylation state or status in the amplified DNA products may then be analyzed by primer extension reaction by using both tagged reverse primers, dNTPs or ddNTPs. Preferably, the dNTPs, ddNTPs or reverse primers that are incorporated into the extension products can be labeled with a detectable label. The detectable label can comprise a radiolabel, a fluorescent label, a luminescent label, an antibody linked to a nucleotide that can be subsequently detected, a hapten linked to a nucleotide that can be subsequently detected, or any other nucleotide or modified nucleotide that can be detected either directly or indirectly.

In a further preferred embodiment, the present invention also provides determining the differential methylation levels of the candidate CpG sites in disease cells by means of high throughput (on microarrays). Microarray based analysis of the relative methylation levels enables working with hundreds of thousands of CpG sites simultaneously rather than one or a few CpG sites at a time. A DNA microarray is composed of an ordered set of DNA molecules of known sequences usually arranged in rectangular configuration in a small space such as 1 cm² in a standard microscope slide format. For example, an array of 200×200 would contain 40,000 spots with each spot corresponding to a probe of known sequence. Such a microarray can be potentially used to simultaneously monitor the expression of 40,000 nucleic acids in a given cell type under various conditions. The probes usually take the form of cDNA, ESTs or oligonucleotides. Most preferred are ESTs and oligonucleotides in the range of 30-200 bases long as they provide an ideal substrate for hybridization. There are two approaches to building these microarrays, also known as chips, one involving covalent attachment of pre-synthesized probes; the other involving building or synthesizing probes directly on the chip. The sample or test material usually consists of nucleic acids that have been amplified by PCR. PCR serves the dual purposes of amplifying the starting material as well as allowing introduction of fluorescent tags. For a detailed discussion of microarray technology, see e.g., Graves, Trends Biotechnol. 17: 127-134 (1999).

Methylation can also be detected by means of high-density microarrays. High-density microarrays are built by depositing an extremely minute quantity of DNA solutions at precise location on an array using high precision machines, a number of which are available commercially. An alternative approach pioneered by Packard Instruments, enables deposition of DNA in much the same way that ink jet printer deposits spots on paper. High-density DNA microarrays are commercially available from a number of sources such as Affymetrix, Incyte, Mergen, Genemed Molecular Biochemicals, Sequenom, Genomic Solutions, Clontech, Research Genetics, Operon and Stratagene. Currently, labeling for DNA microarray analysis involves fluorescence, which allows multiple independent signals to be read at the same time. This allows simultaneous hybridization of the same chip with two samples labeled with different fluorescent dyes. The calculation of the ratio of fluorescence at each spot allows determination of the relative change in the expression of each gene, or the relative methylation level herein, under two different conditions. For example, comparison between a normal tissue and a corresponding tumor tissue using the approach helps in identifying genes whose expression is significantly altered. Thus, the method offers a particularly powerful tool when the gene expression profile of the same cell is to be compared under two or more conditions. High-resolution scanners with capability to monitor fluorescence at various wavelengths are commercially available.

For purposes of detecting large numbers of CpG sites, mixtures of products from different CpG sites using various methylation detection methods as discussed herein, are applied to a microarray, with each CpG site corresponding to a particular location on the microarray. The signal intensity of the products at a particular location can be then determined with methods well known in the art, and the relative methylation levels at those CpG sites can be calculated by comparing the signal intensity at two locations on the microarray corresponding to the methylation and unmethylation states of one particular CpG site.

Table 3 discloses a representative number of down-regulated marker genes whose CpG sites are shown to be differentially methylated in disease.

TABLE 3
Sequences selected for verification of
methylation status in colorectal cancer

| | | SEQ ID |
|---|---|---|
| Gene | Product | NO |
| MMP28 | matrix metallo-proteinase 28 | 134 |
| SLC4A4 | solute carrier family 4, sodium bicarbonate | 151 |
| | cotransporter, member 4 | |
| PYY | peptide YY | 130 |
| SST | somatostatin | 147 |
| PDE9A | phosphodiesterase 9A | 137 |
| CHGA | chromogranin A (parathyroid secretory protein | 144 |
| LOC63928 | hepatocellular carcinoma antigen gene 520 | 145 |
| SCNN1B | sodium channel, nonvoltage-gated 1, beta (Liddle | 146 |
| | syndrome) | |
| CA4 | carbonic anhydrase IV | 138 |
| CA2 | carbonic | 164 |
| | anhydrase II | |
| FCGBP | Fc fragment of IgG binding protein | 165 |
| CKBB | creatine kinase, brain | 171 |
| CES2 | carboxylesterase 2 (intestine, liver) | 172 |
| MT1X | metallothionein 1X | 173 |
| MT2A | metallothionein 2A | 174 |
| FHL1 | four and a half LIM domains 1 | 175 |
| STK39 | serine threonine kinase 39 | 176 |
| SFN | stratifin | 177 |
| GPX3 | glutathione peroxidase 3 | 178 |

V Selection of CpG Sites

In another aspect, the present invention pertains to selection of CpG sites within the CpG islands of the down-regulated marker sequences that can be used in diagnostic, prognostic, and therapeutic assays for detecting a disease, preferably cancer. Generally, the selection comprises the steps of (1) determining the functional recovery of the down-regulated marker sequences containing the methylated CpG sites after demethylation treatment, and (2) validating the CpG sites on the nucleic acid marker sequences in clinical samples. Recently, the abnormal methylation of CpG sites has emerged as a significant mechanism of gene inactivation, particularly tumor suppressor gene inactivation, in cancer. Therefore, the CpG sites whose hypermethylation strongly correlates with disease conditions have significant clinical applications.

In the first step, identifying the CpG sites on the down-regulated marker sequences with great potential for diagnostic utility includes determining whether the methylated CpG sites would show functional recovery of the nucleic acid sequences containing the CpG sites after demethylation treatment. The term “functional recovery” by its ordinary meaning, is meant that the sequences containing the CpG sites go back to at least partially normal function. The term “functional recovery” also means that the expression levels of the nucleic acid sequences containing the CpG sites go back to normal levels, with the levels being manifested at both nucleic acid and protein levels. For example, in one embodiment, functional recovery would mean a significant increase in the nucleic acid expression levels of the nucleic acid sequences containing the CpG sites selected in step one after demethylation treatment. The term “significant increase in the nucleic acid expression levels” as used herein, refers to an increase in nucleic acid expression levels by at least about 10%, preferably at least about 15%, about 25%, about 30%, about 40%, about 50%, about 65%, about 75%, about 85%, about 90%, about 95% or greater. Preferably, the nucleic acid expression levels are determined by measuring the RNA levels of the nucleic acid sequences containing the CpG sites. In another embodiment, functional recovery after demethylation treatment would also result in a significant increase in the levels of the proteins encoded by the down-regulated marker sequences containing the CpG sites after demethylation treatment. The term “significant increase in the levels of the proteins” as used herein, refers to an increase in protein levels by at least about 15%, preferably at least about 25%, 35%, 50%, or greater.

In yet another embodiment, functional recovery would also mean a significant restoration of functional phenotypes involving the functionality of the proteins encoded by the sequences containing the CpG sites selected in step one. The CpG sites that show functional recovery after the demethylation treatment are preferably selected for.

In association with the first step of identifying the CpG sites with great potential for diagnostic utility, a demethylation agent is used to treat the cells or tissues. In a preferred embodiment, the demethylation agent is 5-aza-deoxycytidine. In another preferred embodiment, the concentration of 5-aza-deoxycytidine is in the range of about 1 μM to about 10 μM. The degree of demethylation is determined by any of the methylation assays as described in the previous sections. Preferably, about 30%, more preferably about 40%, or about 50%, or about 60%, or about 75%, or greater reduction in methylation after the demethylation treatment is selected for further assaying the functional recovery.

Furthermore, in association with the first step of identifying the CpG sites with great diagnostic utility, the functional recovery of the nucleic acid sequences containing the CpG sites is analyzed at the nucleic acid level. That is, the nucleic acid expression levels prior to and after the demethylation treatment are determined and compared with each other either qualitatively or quantitatively. In determining the nucleic acid expression levels, various methods may be employed. These methods generally include the steps of contacting the sample derived from the demethylation treated cells or tissues, with probe, hybridizing, and detecting hybridized probe, but using more quantitative methods and/or comparisons to standards. The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements.

In one embodiment, reverse transcription PCR (RT-PCR) is performed using primers designed to specifically hybridize to a predetermined portion of mRNA sequences. Generation of a PCR product by such a reaction is thus indicative of the presence of the nucleic acid sequences in the sample. The technique of designing primers for PCR amplification is well known in the art. Oligonucleotide primers and probes are about 5 to about 100 nucleotides in length, ideally from 17 to 40 nucleotides, although primers and probes of different length are of use. Primers for amplification are preferably about 17-25 nucleotides. Primers useful according to the invention are also designed to have a particular melting temperature (Tm) by the method of melting temperature estimation. Commercial programs, including Oligo™ (MBI, Cascade, Colo.), Primer Design and programs available on the Internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 75° C. and more preferably between about 50 and 65° C. Preferably, the Tm of a probe useful according to the invention is 3-5° C. higher than the Tm of the corresponding amplification primers. It is preferred that, following generation of cDNA by RT-PCR, the cDNA fragment is cloned into an appropriate sequencing vector, such as a PCRII vector (TA cloning kit; Invitrogen). The identity of each cloned fragment is then confirmed by sequencing in both directions. It is expected that the sequence obtained from sequencing would be the same as the known sequences of the marker sequences as described herein.

Alternatively, the nucleic acid expression levels may be detected by Northern analysis. Also alternatively, the nucleic acid expression levels may be determined using the TaqMan™ (Perkin-Elmer, Foster City, Calif.) technique, which is performed with a transcript-specific antisense probe (i.e., a probe capable of specifically hybridizing to the sequences containing the CpG sites). This probe is prepared with a quencher and fluorescent reporter probe complexed to the 5′ end of the oligonucleotide. Different fluorescent markers can be attached to different reporters, allowing for measurement of two products in one reaction (e.g., measurement of the marker sequence). When Taq DNA polymerase is activated, it cleaves off the fluorescent reporters by its 5′-to-3′ nucleolytic activity. The reporters, now free of the quenchers, fluoresce. The color change is proportional to the amount of each specific product and is measured by fluorometer; therefore, the amount of each color can be measured and the RT-PCR product can be quantified. The PCR reactions can be performed in 96 well plates so that samples derived from many individuals can be processed and measured simultaneously. The TaqMan™ system has the additional advantage of not requiring gel electrophoresis and allows for quantification when used with a standard curve.

In one embodiment, the nucleic acid expression levels can be determined by using methods of microarrays such as a DNA chip in an organized array. Oligonucleotides can be bound to a solid support by a variety of processes, including lithography. These nucleic acid probes comprise a nucleotide sequence at least about 8 nucleotides in length, preferably at least about 12 preferably at least about 15 nucleotides, more preferably at least about 25 nucleotides, and most preferably at least about 40 nucleotides, and up to all or nearly all of a sequence which is complementary to at least a portion of the coding sequence of the genes containing the CpG sites to be analyzed. In some embodiments, the microarrays comprise at least 3, 4, 5, 6, 7, 8, 9, 10, 11, 12, 13, 14, or 15, or more nucleic acids that are complimentary to at least a portion of the coding sequences of the genes containing the CpG sites to be analyzed. The present invention provides significant advantages over the available tests for various diseases including cancers, such as colon cancer, because it increases the reliability of the test by providing an array of nucleic acid markers on a single chip.

In particular, the method includes obtaining a biopsy, which is optionally fractionated by cryostat sectioning to enrich tumor cells to about 80% of the total cell population. The DNA or RNA is then extracted, amplified, and analyzed with a DNA chip to determine the presence of absence of the marker nucleic acid sequences.

In one embodiment, the nucleic acid probes are spotted onto a substrate in a two-dimensional matrix or array. Samples of nucleic acids can be labeled and then hybridized to the probes. Double-stranded nucleic acids, comprising the labeled sample nucleic acids bound to probe nucleic acids, can be detected once the unbound portion of the sample is washed away.

The nucleic acid probe can be spotted on substrates including glass, nitrocellulose, etc. The probes can be bound to the substrate by either covalent bonds or by non-specific interactions, such as hydrophobic interactions. The sample nucleic acids can be labeled using radioactive labels, fluorophores, chromophores, etc.

In a preferred embodiment, Affymetrix microarrays are employed to determine the nucleic acid expression levels for the purpose of selecting the CpG sites showing great potential for diagnostic utility.

Furthermore, in association with the first step of identifying the CpG sites with great diagnostic utility, the functional recovery of the genes containing the CpG sites is analyzed at the protein level. That is, the protein levels prior to and after the demethylation treatment are determined and compared with each other either qualitatively or quantitatively. In determining the protein level, the method includes but not limited to, competitive and non-competitive assay systems using techniques such as western blots, radioimmunoassays, ELISA (enzyme linked immunosorbent assay), “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). The protein levels determined by the above methods may be used to correlate with the methylation levels of the selected CpG sites, and in turn with the disease conditions, or progression of the disease conditions.

In the second step, the validation of the CpG sites selected by the methods of the first step comprises determining correlation of the methylation of the CpG sites with a disease in clinical samples. Preferably, the correlation is determined by detecting the methylation of the CpG sites in clinical samples obtained from a subject having or suspected of having a disease to be detected compared to that in a normal sample. In the case of determining correlation between a specific CpG site and a disease, a good correlation between the methylation at this specific CpG site and a disease could mean that the CpG site shows a significant increase in methylation in disease samples as compared to that in normal, disease-free samples. The CpG sites that show a significant increase in methylation in diseased samples as compared to that in normal, disease-free samples are preferably selected. In one preferred embodiment, the increase in methylation of the CpG sites in disease cells or tissue are preferably at least about 1.5 fold, more preferably 2 fold, over that in normal cells or tissues.

In addition, a good correlation between the methylation at a specific CpG site on a nucleic acid marker sequences and a disease could also mean that the degree of methylation at the CpG site shows distinct differences at different stages of a disease. For example, the methylation at the specific CpG site could change as the disease progresses to higher stages.

A good correlation could also encompass the relationship between multiple CpG sites on a single nucleic acid marker sequence and a disease. In this regard, the methylation of multiple CpG sites on one nucleic acid marker sequence could be determined to establish the correlation between said multiple CpG sites and the disease. For example, for one specific disease to be assayed, the methylation at one or more CpG sites on a single nucleic acid marker sequence could either increase or decrease as the disease progresses to advanced stages. Alternatively, either increased number of or decreased number of CpG sites on a single nucleic acid marker sequence could be methylated as the disease progresses to advanced stages.

Furthermore, based on the good correlation between methylation at the one or more specific CpG sites and a disease, one of skill in the art could establish methylation pattern or fingerprints at said CpG sites corresponding to the disease or the stages of the disease. Such methylation pattern or fingerprints provides for an accurate clinical assessment of the disease in a subject by determining the methylation state of said CpG sites in a sample obtained from the subject.

The methylation levels of the CpG sites in clinical samples may be determined by methods known in the art, or the methods described above in section V. In one preferred embodiment, the MSP method is employed for this purpose. In another preferred embodiment, the bisulfite genomic sequencing method is employed. In yet another preferred embodiment, the MSPE method is employed. In a further preferred embodiment, the high throughput or microarray methods are employed. The CpG sites that show signification methylation in the disease such as cancer or tumor as compared to the normal adjacent tissue are selected. See Examples 4 and 5 for representative CpG sites showing great diagnostic utility. Table 4 lists non-limiting examples of cell lines used for verification of methylation.

TABLE 4
Cell lines used for verification of methylation

| Name | Source | Tumorigenic | Culture Media | Conditions |
|---|---|---|---|---|
| SW480 | primary adenocarcinoma | yes | Leibovitz's L-15 | 5 μM 5-aza-2′- |
| | | | medium with 2 mM | deoxycytidine for 3 |
| | | | L-glutamine, 90% | days |
| | | | fetal calf serum | |
| SW620 | recurrence of adenocarcinoma | yes | Leibovitz's L-15 | 5 μM 5-aza-2′- |
| | (same patient as for SW480) | | medium with 2 mM | deoxycytidine for 5 |
| | | | L-glutamine, 90% | days |
| | | | fetal calf serum | |
| LS123 | primary adenocarcinoma | no | Eagle's MEM | 1 μM 5-aza-2′- |
| | | | medium with 15% | deoxycytidine for 3 |
| | | | fetal calf serum | days |
| LS174T | primary adenocarcinoma | yes | Eagle's MEM | 3 μM 5-aza-2′- |
| | | | medium with 10% | deoxycytidine for 5 |
| | | | fetal calf serum | days |
| HT-29 | primary adenocarcinoma | yes | McCoy's 5a | 5 μM 5-aza-2′- |
| | | | medium with 1.5 mM | deoxycytidine for 5 |
| | | | L-glutamine | days |
| | | | and 10% fetal calf | |
| | | | serum | |

VI Use of the CpG Sites for Diagnosis, Prognosis, Staging, and Monitoring of Therapy

In all the methods described in the present invention, the identification of sequences that are abnormally methylated is used for identifying a disease, disease state, or premalignant conditions. Such disease or disease state or premalignant conditions include cancer, multiple sclerosis, Alzheimer's disease, Parkinson's disease, depression and other imbalances of mental stability, atherosclerosis, cystic fibrosis, diabetes, obesity, Crohn's disease, and altered circadian rhythmicity, arthritis, inflammatory reactions or disorders, psoriasis and other skin diseases, autoimmune diseases, allergies, hypertension, anxiety disorders, schizophrenia and other psychoses, osteoporosis, muscular dystrophy, amyotrophic lateral sclerosis and circadian rhythm-related conditions. Preferably, the diseases that have been shown to be strongly associated with aberrant methylation include cancer. Examples of cancer include but not limited to, adenocarcinoma, lymphoma, blastoma, melanoma, sarcoma, and leukemia. More particularly, examples of cancer also include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Preferably, the cancers include breast, colon, and lung cancer.

The determination of the methylation level of one or more selected CpG sites within one or more marker sequences in a patient as compared to a normal individual, provides a means of diagnosing or monitoring the patient's disease status, and/or patient response or benefit to therapy. In one aspect, the present invention provides methods for detecting disease such as cancer, or alternatively, determining whether a subject is at risk for developing disease such as cancer by detecting the methylation level of one or more selected CpG sites, wherein the methylation level of the CpG sites correspond to a particular disease or condition. In a preferred embodiment, the cancer is colon cancer, and the CpG sites are the ones as selected by the method discussed in the previous sections.

In clinical applications, human tissue samples can be screened for the hypermethylation of one or more CpG sites selected by the methods of the present invention. Such samples may comprise tissue samples, whole cells, cell lysates, or isolated nucleic acids, including, for example, needle biopsy cores, surgical resection samples, lymph node tissue, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art. The methylation levels of the selected CpG sites in these samples would be compared with statistically valid groups of metastatic, non-metastatic malignant, benign, or normal colon tissue samples.

In one embodiment, the diagnostic method comprises determining whether a subject has increased methylation levels of the selected CpG sites. The method comprises determining the methylation levels of the selected CpG sites by using the methylation methods discussed herein. Specifically, the method comprises:
(a) determining the degree of methylation of one or more CpG sites on nucleic acid sequences in a biological sample obtained from the subject;
(b) determining the presence of, predisposition to, or stage of the disease in the subject based on the degree of methylation.

In another embodiment, the present invention provides methods for determining disease prognosis and stage based on examining the methylation levels of the selected CpG sites within one or more marker sequences using the methods described in the present invention. If disease is detected in a subject using a technique other than by determining the methylation levels of the selected CpG sites, then the differential methylation levels of the selected CpG sites within the marker sequences can be used to determine the prognosis and stage for the subject. In general, methods used for prognosis or stage of a disease involve comparison of the methylation levels or extents of selected CpG sites in a sample of interest with that of a control to detect relative differences in the methylation levels, wherein the difference can be measured qualitatively and/or quantitatively. For example, the methylation levels of the selected CpG sites can be compared with the methylation levels of the same CpG sites in disease free or normal samples. Alternatively, the methylation levels of the selected CpG sites can also be compared with the methylation levels of the same CpG sites observed in various stages of disease. Alternatively, the methylation levels of the selected CpG sites can also be compared with the methylation levels of the same CpG sites determined from a sample at an earlier point in time from the same patient. Preferably, the disease is cancer. More preferably, the cancer is colon cancer, and the marker sequences are the ones identified in Tables 6, 7, and 8.

In one embodiment, the methods comprise:
(a) detecting in a biological sample of the subject at a first point in time, the degree of methylation of one or more CpG sites on nucleic acid sequences, wherein the CpG sites are differentially methylated at different stages of the disease;
(b) repeating step (a) at a subsequent point in time; and
(c) comparing the degree of methylation of the CpG sites in step (a) and (b), wherein a change in the degree of methylation is indicative of disease progression in the subject.

In another embodiment, the present invention also provides methods that permit the assessment and/or monitoring of patients who will be likely to benefit from both traditional and non-traditional treatments and therapies for disease such as cancer, particularly colon cancer. The present invention thus embraces testing, screening and monitoring of patients undergoing anti-disease treatments and therapies, used alone, in combination with each other, and/or in combination with anti-disease drugs, anti-neoplastic agents, chemotherapeutics and/or radiation and/or surgery, to treat patients.

Particularly, the method including determining the efficacy of a test compound for inhibiting a disease in a subject, wherein the method comprises:
(a) detecting in a first biological sample of the subject, the degree of methylation of one or more CpG sites, wherein the sample has not been exposed to the test compound, and wherein the CpG sites are methylated in the disease;
(b) detecting in a second biological sample of the subject, the degree of methylation of the same CpG sites, wherein the sample has been exposed to the test compound; and
(c) comparing the degree of methylation of the CpG sites in step (a) and (b), wherein a decrease in methylation after the sample has been exposed to the test compound, is indicative of the efficacy of the test compound.

An advantage of the present invention is the ability to monitor, or screen over time, those patients who can benefit from one, or several, of the available therapies, and preferably, to monitor patients receiving a particular type of therapy, or a combination therapy, over time to determine how the patient is faring from the treatment(s), if a change, alteration, or cessation of treatment is warranted; if the patient's disease has been reduced, ameliorated, or lessened; or if the patient's disease state or stage has progressed, or become metastatic or invasive. The treatments for cancer embraced herein also include surgeries to remove or reduce in size a tumor, or tumor burden, in a patient. Accordingly, the methods of the invention are useful to monitor patient progress and disease status post-surgery.

The identification of the correct patients for a therapy according to this invention can provide an increase in the efficacy of the treatment and can avoid subjecting a patient to unwanted and life-threatening side effects of the therapy. By the same token, the ability to monitor a patient undergoing a course of therapy using the methods of the present invention can determine whether a patient is adequately responding to therapy over time, to determine if dosage or amount or mode of delivery should be altered or adjusted, and to ascertain if a patient is improving during therapy, or is regressing or is entering a more severe or advanced stage of disease, including invasion or metastasis, as discussed further herein.

A method of monitoring according to this invention reflects the serial, or sequential, testing or analysis of a patient by testing or analyzing the patient's body fluid sample over a period of time, such as during the course of treatment or therapy, or during the course of the patient's disease. For instance, in serial testing, the same patient provides a body fluid sample, e.g., serum or plasma, or has sample taken, for the purpose of observing, checking, or examining the methylation levels of one or more of the CpG sites of the invention in the patient during the course of treatment, and/or during the course of the disease, according to the methods of the invention.

Similarly, a patient can be screened over time to assess the differential methylation levels of one or more selected CpG sites within the marker sequences in a body fluid sample for the purposes of determining the status of his or her disease and/or the efficacy, reaction, and response to disease including cancer or neoplastic disease treatments or therapies that he or she is undergoing. It will be appreciated that one or more pretreatment sample(s) is/are optimally taken from a patient prior to a course of treatment or therapy, or at the start of the treatment or therapy, to assist in the analysis and evaluation of patient progress and/or response at one or more later points in time during the period that the patient is receiving treatment and undergoing clinical and medical evaluation.

In monitoring a patient's methylation levels of the selected CpG sites of the invention over a period of time, which may be days, weeks, months, and in some cases, years, or various intervals thereof, the patient's body fluid sample, e.g., a serum or plasma sample, is collected at intervals, as determined by the practitioner, such as a physician or clinician, to determine the levels of one or more of the markers in the patient compared to the respective levels of one or more of these analytes in normal individuals over the course or treatment or disease. For example, patient samples can be taken and monitored every month, every two months, or combinations of one, two, or three month intervals according to the invention. Quarterly, or more frequent monitoring of patient samples, is advisable.

The differential methylation levels of the one or more CpG sites within the marker sequences found in the patient are compared with the respective methylation levels of the same CpG sites in normal individuals, and with the patient's own methylation levels, for example, obtained from prior testing periods, to determine treatment or disease progress or outcome. Accordingly, use of the patient's own methylation levels monitored over time can provide, for comparison purposes, the patient's own values as an internal personal control for long-term monitoring of methylation levels, and thus disease presence and/or progression. As described herein, following a course of treatment or disease, the determination of an increase or decrease in methylation levels of the selected CpG sites in a patient over time compared to the respective methylation levels of the same CpG sites in normal individuals reflects the ability to determine the severity or stage of a patient's disease, or the progress, or lack thereof, in the course or outcome of a patient's therapy or treatment.

In monitoring a patient over time, a reduction in the methylation levels of the selected CpG sites from increased levels compared to normal range values at or near to the levels of the analytes found in normal individuals is indicative of treatment progress or efficacy, and/or disease improvement, remission, tumor reduction or elimination, and the like.

As will be understood by the skilled practitioner in the art, the monitoring method according to this invention is preferably, performed in a serial or sequential fashion, using samples taken from a patient during the course of disease, or a disease treatment regimen, (e.g., after a number of days, weeks, months, or occasionally, years, or various multiples of these intervals) to allow a determination of disease progression or outcome, and/or treatment efficacy or outcome. If the sample is amenable to freezing or cold storage, the samples may be taken from a patient (or normal individual) and stored for a period of time prior to analysis.

The present invention also includes a method of assessing the efficacy of a test composition for inhibiting diseases such as cancers, or colon cancer. As described above, differential methylation levels of the selected CpG sites within the marker sequences of the invention correlate with the disease state of disease cells, particularly cancer cells, more particularly colon cancer cells. It is recognized that changes in the methylation levels of the selected CpG sites within the marker sequences of the present invention result from the disease state of cells. Thus, compositions which inhibit disease in a patient will cause the methylation levels of the selected CpG sites within the marker sequences to change to a level near the normal level for the marker sequences. The method thus comprises comparing methylation levels of the selected CpG sites within one or more marker sequences in a first biological sample maintained in the presence of a test composition with those of the same CpG sites in a second biological sample maintained in the absence of the test composition. A significant difference in the methylation levels of the selected CpG sites within one or more marker sequences is an indication that the test composition inhibits the disease. In a preferred embodiment, the cancer is colon cancer. In another embodiment, the cell samples may be aliquots of a single sample obtained from either a healthy subject or a patient with disease conditions.

VII Kits

The present invention also provides kits for practicing the use of the selected CpG sites in the diagnosis, prognosis, or staging of a disease, or monitoring of therapy. The kits may comprise a bisulfite-containing reagent that modifies the unmethylated cytosine, as well as oligonucleotides for determining the methylation state of one or more specific CpG sites on a specific nucleic acid marker sequence. Determining the methylation state may comprise one or more of the following techniques: methylation-specific PCR, bisulfite genomic sequencing methods, methylation-specific primer extension methods, and all other methods known in the art for determining CpG methylation. The oligonucleotides could encompass the primers used for amplifying the bisulfite-treated nucleic acids, wherein the amplification can employ any method known in the art. Additionally, oligonucleotides could also encompass the primers or probes used in measuring and/or quantifying the methylation of the CpG sites. Preferably, the oligonucleotides comprise at least about 7, 15, 20, 25, 30, 50, 75, 100, 125, 150, 175, 200, 250, 300, 350, or more consecutive nucleotides in length. More preferably, the oligonucleotides comprise about 8 to 60 consecutive nucleotides in length. More preferably, the oligonucleotides could be modified with non-nucleotide moieties. For example, the oligonucleotides could have altered sugar moieties, altered bases, both altered sugars and bases or altered inter-sugar linkages. Probes may be complementary to a position on the sequence of the nucleic acid marker sequences identified using the claimed method. Preferably, the probes that are complementary to a region on the nucleic acid marker sequences are used for detecting and/or quantifying either methylated or unmethylated nucleic acid marker sequences. For example, the probes may be designed to hybridize under stringent or moderately stringent conditions, to either methylated or unmethylated nucleic acid marker sequences listed in Tables 1, or 3, or 5. Also preferably, the probes may be conjugated with a detectable label.

The kits may also comprise a set of control/reference values indicating normal and various clinical progression stages of a disease. In one embodiment, the set of control/reference values is indicative of various clinical progression stages of cancer. In a preferred embodiment, the set of control/reference values is indicative of various clinical progression stages of colon cancer. Moreover, a kit may also comprise positive controls, and/or negative controls for comparison with the test sample. A negative control may comprise a sample that does not have any nucleic acid marker sequences. A positive control may comprise various degrees of methylation at one or more specific CpG sites. A kit may further comprise instructions for carrying out and evaluating the results.

### Examples

### Example 1

### Gene Expressing Profiling

Twenty well characterized, microdissected samples of colorectal cancer tissue were obtained from consenting patients. A second set of twenty, microdissected samples of normal adjacent colon tissue were also obtained. Total RNA was extracted from these samples using RNeasy kits (QIAGEN, Valencia, Calif.) according to the manufacturer's instructions. Expression profiling was performed using the GeneChip expression arrays from Affymetrix (Santa Clara, Calif.). Reverse transcription, second-strand synthesis, and probe generation was accomplished by standard Affymetrix protocols. The Human Genome U133A GeneChip, which contains more than 15,000 substantiated human genes, was hybridized, washed, and scanned according to Affymetrix protocols. Changes in cellular mRNA levels in the cancerous tissues were compared with mRNA levels in the normal colon tissues. GeneSpring v4.2 (Silicon Genetics, Redwood City, Calif.) was used to normalize and scale results and compare gene expression levels in the cancer tissue relative to that in the normal tissue.

Applying a set of filters to the normalized data identified the down-regulated genes in the cancer samples. First, a non-parametric test defined the genes that were statistically associated with either the cancer or the normal samples. From this set, the genes with normalized signals of 5 or greater in any one of the normal samples were selected. To further reduce the set, the genes with normalized signals greater than 5 in any of the cancer samples were identified and removed. Finally, using the Affymetrix absent/present calls, those genes that were not present in at least five of the twenty normal samples were removed. Table 1 shows the candidate genes identified using this process.

### Example 2

### Identification of CpG Sites

From this list of genes in Table 1, the subset of genes (Table 2) containing at least one CpG island in the published sequence of the promoter-first exon region (1000 bp upstream and 500 bp down stream from exon 1) was identified. The standard definition of a CpG island (having regions of DNA greater than 200 bp, with a guanine/cytosine content above 0.5 and an observed or an expected presence of CpG above 0.6) was used. Genes were initially examined in the UCSC Genome Browser for the presence of CpG island(s) in the 5′ region. Sequences were then analyzed in the Cpgplot program to verify the presence of island(s) in the defined region (1000 bp upstream and 500 bp down stream from exon 1).

### Example 3

### Verification of Methylation by Bisulfite Sequencing

Samples: Paired tumor and adjacent normal tissues from twelve colorectal cancer patients were collected under institutional review board (IRB) approval with patient consent. Tissues were flash frozen in LN₂ and stored at −80° C. prior to DNA extraction. All tissues were blinded.

Cell lines: A panel of five colorectal cancer cell lines was used. Cells were grown to ˜50% confluence in the appropriate culture medium prior to treatment with 5-aza-2′-deoxycytidine. Optimal concentrations and incubation times (Table 4) were determined by assaying for reduction of p 16 promoter methylation using MSP. Cells were harvested, pelleted by centrifugation, and washed twice in Hanks buffered saline solution. Cell pellets were stored at −80° C. Control cells were maintained simultaneously without 5-aza-2′-deoxycytidine treatment.

DNA extraction: DNA was purified from tissues and cell lines using the QIAGEN DNeasy® Tissue Kit. Approximately 25-35 mg of each tissue was pulverized under liquid nitrogen before extraction. Elution volume for tissues was 200 μL. A final volume of 200 μL of cell line DNA was extracted from 15 to 25 μL of each packed cell pellet (between 10⁶-10⁷ cells). Purified DNA was stored at −20° C.

Bisulfite modification: Modification was performed according to the Frommer method (See Frommer M, et al., PNAS, 89: 1827-1831 (1992).) One μg genomic DNA was diluted into 50 μl with distilled H₂O, 5.5 μl of 2M NaOH was added, and the mixture incubated at 37° C. for 10 minutes (to create single stranded DNA). Thirty μl of freshly prepared 10 mM hydroquinone (Sigma) was added to each tube. Five hundred twenty μl of freshly prepared 3M sodium bisulfite (Sigma S-8890), pH 5.0 was then added. Reagents were thoroughly mixed and then covered with mineral oil and incubated at 50° C. for 16 hours. After removing the oil, 1 ml of Wizard DNA Cleanup Resin (Promega A7280) was added to each tube prior to applying the mixture to miniprep column in the DNA Wizard Cleanup kit. The column was washed with 2 ml of 80% isopropanol, and eluted with 50 μl of heated water (60-70° C.). 5.5 μl of 3 M NaOH to was added to each tube, and incubated at room temperature for 5 minutes. Then 1 μl glycogen was added as carrier, 33 μl of 10 M NH₄Ac, and 3 volumes of ethanol for DNA precipitation. The pellet was spun down and washed with 70% ethanol, dried and resuspended in 20 μl water. In some instances, the EZ DNA Methylation Kit (Zymo Research) which uses a simplified version of the Frommer method was used. In these cases, 1 μg of genomic DNA was denatured in 0.3M NaOH for 15 minutes at 37° C. followed by incubation at 50° C. for 16 hours in 0.5 mM hydroquinone and a saturated solution of sodium bisulfite at pH 5. Modified DNA was bound to the Zymo column membrane, then desulfonated with 0.3M NaOH for 15 minutes at room temperature. DNA was washed and resuspended with 50 μL 10 mM Tris-HCl-0.1 mM EDTA, pH 7.5 and stored at −20° C. The bisulfite reaction results in conversion of an umethylated cytosine to uracil. Methylated cytosine remains unchanged after the bisulfite reaction. The resulting bisulfite modified DNA is single stranded.

PCR amplification for sequencing: Primers were designed to amplify both methylated and unmethylated fragments of DNA (Table 5). Five μL of modified DNA (1/10 of modification reaction) was amplified first in a 25 μL reaction volume containing 10 mM Tris-HCl pH8.3, 50 mM KCl, 1.5 mM to 2 mM MgCl2, (Applied Biosystems), 0.25 mM each dNTP, 0.5 unit AmpliTaq (Applied Biosystems), and sequencing primers (each at 200 nM). Cycling conditions were 10 minutes at 95° C., 40 cycles of 30 seconds at 95° C., 30 seconds at 54-62° C., 30 seconds at 72° C., subsequently followed by extension for 5 minutes at 72° C.

Reaction products were purified either by the shrimp-alkaline phosphatase-Exol standard method or on the Qiagen Qiaquick PCR clean-up column and eluted in 30 μL 10 mM Tris-HCl, pH8.5. The amount of DNA was determined by absorbance at OD₂₆₀ and stored at −20° C. before sequencing. Purified amplicons were sequenced by the chain-termination sequencing method. Reverse sequencing primers at 3.2 μM concentration and 200 ng of each purified amplicon diluted in 10 μL dH2O were sent to a commercial sequencing service (SeqWright).

Vector NTI ContigExpress (Informax, Inc.) was used to align sequences. Methylated CpG sites were determined by comparing the peak height of C and T traces at each CpG. A C-trace peak height to T-trace peak height ratio of >0.5 indicates a methylated site.

$$\frac{C - trace peak height}{T - trace peak height} > 0.5 = Methylated$$

TABLE 5
Primers for sequencing reactions

| | | | | | | Sequence |
|---|---|---|---|---|---|---|
| | Primer | Forward/ | Primer Sequences | | Amplicon | ID |
| Gene name | no. | reverse | 5′-3′ | Tₘ° C. | length | number |

| | | | | | | |
|---|---|---|---|---|---|---|
| SLC4A4 | 63 | F | GGTAGTGGTAGTGGTYGTTGTAGT TT | 75.8 | 222 | 179 |
| | 64 | R | CCRCAATTAACCTCTCTCTCC | 73.4 | | 180 |
| PYY | 77 | F | GGGGAGGTAGGTAGGGTTTATGT | 77.3 | 290 | 181 |
| | 78 | R | CAACRCCCCTAAACAAACRAACAA | 72.2 | | 182 |
| LOC63928a | 51 | F | YGTTTTGGGGTTGGGAGYGTT | 73.4 | 341 | 183 |
| | 52 | R | RCRTTCTCTCCTCCCRCCRAAA | 73.6 | | 184 |
| LOC63928b | 53 | F | GGGGTTATTGGGGYGGTTAYGT | 75.4 | 227 | 185 |
| | 54 | R | TCCCTAACCCCAAACRCCTAAA | 73.6 | | 186 |
| SCNN1B | 49 | F | TTGTAGGGGTGTGGATGTGAT | 73.4 | 358 | 187 |
| | 50 | R | AACTTACTAAACRCTACCRACCTAAC | 72.6 | | 188 |
| CA4-1 | 55 | F | TTTTGYGTATAGGGTAAGAGGTGGTT | 74.2 | 272 | 189 |
| | 56 | R | AACAACATCCRCATCTTACRAAACAA | 71.1 | | 190 |
| CA4-2 | 57 | F | AAATTTAGGTYGGTAGGATYGTTGTAT | 71.3 | 425 | 191 |
| | 58 | R | AAACTCCCAACTCRTCTCRCCRAA | 73.9 | | 192 |
| EDN3 | 155 | F | GGTTTAAAGGTTYGGYGAGGTA | 71.7 | 319 | 193 |
| | 156 | R | AACCCCRACTCCATAAACCTAAATC | 74.1 | | 194 |
| GPX3 | 144 | F | GGAGGTGGGGAGTTGAGGGTA | 79.2 | 221 | 195 |
| | 88 | R | CCTACAACAACCRAACCATAACRAAA | 72.6 | | 196 |
| P16 | 17 | F | GAAGAAAGAGGAGGGGTTGG | 75.2 | 273 | 197 |
| | 18 | R | CTACAAACCCTCTACCCACC | 75.2 | | 198 |
| MMP28 | 65 | F | YGTAGAGTAGTTTTATTTTYGGGGTT | 71.1 | 208 | 199 |
| | 66 | R | RCCTCCTTACRCAACTCCTAA | 71.4 | | 200 |
| CES2a | 211 | F | TTGTTYGGATTYGGGAATATGAT | 70.5 | 338 | 201 |
| | 212 | R | CATTTCACRAACCCCTACCRAT | 65.3 | | 202 |
| CES2b | 213 | F | TTTAAGGTTGGGTAAGGTATTGAT | 68.2 | 279 | 203 |
| | 214 | R | CTCCCAAACRCCTACCCTC | 67.6 | | 204 |
| CA9 | 241 | F | (AGCACCCGGATGGCGTAGA) GGGGA | 77.3 | 316 | 205 |
| | (162) | | GAGGGTATAGGGTTAGATAA | | | |
| | 242 | R | (GAT TGG CGG CAC TGG CTA TC) AAAT | 72.2 | | 206 |
| | (163) | | CCTCCTACATCCRAAACAAC | | | |
| CBFA2T3a | 138 | F | GGGGYGGAGTTGAGYGTTA | 72.9 | 261 | 207 |
| | 139 | R | CCTAAACCATACCRAAAACTCRACT | 72.4 | | 208 |
| CBFA2T3b | 140 | F | TGTGAGTTTTTGTGGAGGGATAGA TG | 75.8 | 222 | 209 |
| | 141 | R | CRACCTCAACCCACAAAATAAATA AA | 71.1 | | 210 |
| CHGA | 94 | F | GGGTTCGTTATGCGTTTCGTC | 75.3 | 234 | 211 |
| (M only) | | | | | | |
| | 95 | R | CCCAAACGAAAACCACACTACAA | 73.8 | | 212 |
| CHGA | 96 | F | GTTTGGTGTTTGGGTTTGTTATGT | 72.2 | 244 | 213 |
| (U only) | | | | | | |
| | 97 | R | CCAAACAAAAACCACACTACAAAATC | 72.6 | | 214 |
| CHGA | 71 | F | GYGAGGGYGTTGTTGTTGTTATYGT | 74.1 | 292 | 215 |
| | 93 | R | ACTCCCCRCRCTCRCTCACCTTA | 77.3 | | 216 |
| ERCC1a | 89 | F | AGAGAGGTYGGAAGTGTTGYGAGTT | 75.7 | 239 | 217 |
| | 90 | R | CCCTCCCCACRCCTAACCTTA | 77.3 | | 218 |
| ERCC1b | 91 | F | GTGGAGATTGGYGTYGYGGAAGTT | 75.6 | 340 | 219 |
| | 92 | R | CRTCTACRTTCTCATCCCRCAACAA | 74.1 | | 220 |
| FANCA | 227 | F | TYGTYGGGAGGAATAGYGGTTGT | 73.0 | 326 | 221 |
| | 228 | R | CCAAACRCRCACACCCRTTAACTAA | 70.9 | | 222 |
| FLJ21511 | 151 | F | AAGGAGGTAAAGGYGGGGATTA | 73.6 | 267 | 223 |
| | 152 | R | AATCRAACCCRCTACCCTAACC | 73.6 | | 224 |
| hMLH1 | 67 | F | GGAGTGAAGGAGGTTAYGGG | 75.2 | 225 | 225 |
| | 68 | R | CCRACCCRAATAAACCCAAC | 71.1 | | 226 |
| HPGDa | 231 | F | TTAGAAYGTTTAGGGGGTAGGTGA | 71.1 | 297 | 227 |
| | 232 | R | CRCCRAACTTACCTTAACRCCCTTA | 66.8 | | 228 |
| HPGDb | 233 | F | YGGYGYGGTTTAGGGTATAGGTAGA | 71.0 | 242 | 229 |
| | 234 | R | TTAAATTCCCTCCCAACCACT | 70.9 | | 230 |
| MGMT | 69 | F | GTTTYGGATATGTTGGGATAG | 69.5 | 251 | 231 |
| | 70 | R | AACACTTAAAACRCACCTAAAA | 66.1 | | 232 |
| MT1G | 134 | F | GYGGGTGTAGTAGGTAATTTTAG | 72.0 | 298 | 233 |
| | 135 | R | AAAACRAAATAAAACCCAACAAC | 66.6 | | 234 |
| MT1X | 239 | F | GGAGAGGGAGAGGTAGGTAATGTT | 71.3 | 263 | 235 |
| | 240 | R | TAATAAAACCCAAAAACCRACRAC T | 65.1 | | 236 |
| PDE9Aa | 61 | F | AGGGGAYGAAATTGTTGAATTTAGT | 70.8 | 378 | 237 |
| | 62 | R | TCCCRATACCCCCTAAACAACTATA | 74.1 | | 238 |
| PDE9Ab | 73 | F | AGTYGATYGGGGGTTGGAGTT | 73.4 | 383 | 239 |
| | 74 | R | TCCCATCCTACRCCCRACRACTA | 75.5 | | 240 |
| PDE9Ac | 75 | F | GGYGTAGGATGGGATTYGGTTT | 73.6 | 542 | 241 |
| | 76 | R | RACCCRAATCCCCCTCTACAA | 73.4 | | 242 |
| PDE9Ad | 73 | F | AGTYGATYGGGGGTTGGAGTT | 73.4 | 272 | 239 |
| | 98 | R | CCRCRACRCTCAACCAACCACAA | 75.5 | | 243 |
| PDE9Ae | 99 | F | GAGYGYGAGTYGAGYGGAGGAGATT | 77.3 | 211 | 244 |
| | 74 | R | TCCCATCCTACRCCCRACRACTA | 75.5 | | 240 |
| SFNa | 243 | F | (AGC ACC CGG ATG GCG TAG A) TG | 74.6 | 337 | 245 |
| | (162) | | GAGAGAGTTAGTTTGATTTAGAAGGTT | | | |
| | 244 | R | (GAT TGG CGG CAC TGG CTA TC) TCCC | 72.4 | | 246 |
| | (163) | | CRACCTCCTTAATAAAATAAC | | | |
| SFNb | 217 | F | TGGAGGGTGTTGTTTAGTATTGAGTA | 71.2 | 234 | 247 |
| | 218 | R | RATAACCACCTCRACCAAATAACRATA | 65.1 | | 248 |
| SLC26A2a | 166 | F | (AGCACCCGGATGGCGTAGA) TTTYGG | 70.2 | 253 | 249 |
| | (162) | | TTTGGGTYGAGTTATTG | | | |
| | 167 | R | (GATTGGCGGCACTGGCTATC) CRTCTT | 72.6 | | 250 |
| | (163) | | CCACCRTAACCTAACTAAAA | | | |
| SLC26A2b | 153 | F | TTTYGGTTTGGGTYGAGTTATTG | 70.2 | 253 | 251 |
| | 154 | R | CRTCTTCCACCRTAACCTAACTAAAA | 72.6 | | 252 |
| SLC26A4a | 219 | F | GGTTGGGAAAGATYGTAGTTTGT | 69.6 | 337 | 253 |
| | 220 | R | AAATCTCTCCCCTCRTCCTATT | 67.7 | | 254 |
| SLC26A4b | 221 | F | YGTTGYGGGAGAGTTTGGTTAAG | 71.6 | 248 | 255 |
| | 222 | R | TAAATTCATTTCRAACCCRAAACTAAT | 65.6 | | 256 |
| SLC5A8a | 223 | F | AGTATTTAGGGTAGYGGGTYGATT | 67.4 | 286 | 257 |
| | 224 | R | CRATACCCCRTAACRTATCCATAA | 64.0 | | 258 |
| SLC5A8b | 225 | F | GYGTAGGGTTTAGGYGATYGTG | 67.4 | 250 | 259 |
| | 226 | R | AAATACCCAAAACAATAACRACTAAC | 64.6 | | 260 |
| SST | 47 | F | GTAAAAGGGTTGGTGAGATTTGG | 73.8 | 343 | 261 |
| | 48 | R | CRAAAAAATCTCCTTACCTACTTCC | 72.4 | | 262 |
| TFEBa | 81 | F | YGTGTTTAGYGGGATTGTAGYGAGAAT | 74.3 | 280 | 263 |
| | 82 | R | CCRCCACCTACTCCCRACCTA | 77.3 | | 264 |
| TFEBb | 83 | F | TTGGTGGTAYGGGGTYGGAGT | 75.3 | 222 | 265 |
| | 84 | R | CCTATCTCCRAAACCCACRAAATAA | 72.4 | | 266 |
| TFEBc | 85 | F | GAGGGTTYGGGATTTTYGATTT | 69.9 | 395 | 267 |
| | 86 | R | CRACCCCAACCRTATCCRATAA | 71.1 | | 268 |

### Example 4

### Functional Selection of the Relevant CpG Sites

Identification of sites within the CpG islands with the greatest potential for diagnostic utility was done by comparing sequencing data for (a) CRC tumor to adjacent normal tissue and (b) cell lines (treated vs. untreated) for 3 genes: SCNN1B, CA4, and GPX3 (Tables 6, 7, and 8). Nucleotides in each amplicon were numbered from the start of the forward primer. The numbers given for CpG sites in Tables 6, 7, and 8 are derived from this ordering. Relevant sites would have greater methylation in the tumor pools and the untreated cell lines than in the adjacent normal tissue pools and treated cell lines. Examples of preferred sites are #192 and #267 SCNN1B; #52 CA4; and #75 and #84 GPX3. Cell line data may vary from tissue data in that cell lines tend to be more highly methylated. As cell lines differ in their susceptibility to demethylation by 5-aza-2′-deoxycytidine, evidence of demethylation in at least one of the cell lines treated was enough to support selection of a relevant site. Relevant sites are included in regions to be detected using methylation-specific PCR, MSPE or other assays that rely on a limited number of sites.

Further support for the clinical importance of these sites comes from the changes seen in gene expression of the genes after treatment of cell lines with 5-aza-2′-deoxyctyidine. These values were obtained from Affymetrix expression profiling of treated and untreated cell lines using the procedure described above. Genes that had at least one cell line that showed a restoration of gene expression of 2-fold or greater after treatment with the demethylating agent were selected. Examples of expression restoration was seen for SCNN1B (cell line LS123 at 4.1-fold), CA4 (cell line at LS174T 2.8), and GPX3 (cell line LS174T at 8.5-fold).

TABLE 6
Sequencing results for SCNN1B on cell lines and CRC tumor/adjacent normal
tissue pools at specific CpG dinucleotides

| | CpG sites | | | | | | | | | | |
|---|---|---|---|---|---|---|---|---|---|---|---|
| Sample Type | #179 | #192 | #203 | #223 | #228 | #230 | #234 | #238 | #245 | #267 | #295 |
| HT29 | 56 | 92 | 36 | 83 | 86 | 79 | 80 | 90 | 77 | 76 | 36 |
| HT29 treated | 70 | 90 | 36 | 79 | 74 | 67 | 69 | 75 | 65 | 55 | 26 |
| SW480 | 93 | 40 | 27 | 95 | 97 | 97 | 97 | 97 | 98 | 95 | 87 |
| SW480 treated | 80 | 44 | 21 | 89 | 83 | 80 | 80 | 87 | 51 | 57 | 69 |
| SW620 | 73 | 94 | 54 | 96 | 97 | 91 | 95 | 99 | 61 | 87 | 3 |
| SW620 treated | 59 | 88 | 30 | 93 | 95 | 94 | 91 | 86 | 26 | 67 | 23 |
| LS174T | 5 | 58 | 32 | 93 | 96 | 96 | 88 | 83 | 84 | 94 | 5 |
| LS174T treated | 7 | 56 | 40 | 75 | 81 | 72 | 70 | 55 | 40 | 47 | 7 |
| LS123 | 49 | 54 | 50 | 95 | 96 | 93 | 93 | 80 | 91 | 56 | 33 |
| LS123 treated | 56 | 63 | 42 | 90 | 87 | 82 | 80 | 77 | 81 | 59 | 23 |
| Early stage normal | 51 | 21 | 30 | 31 | 32 | 24 | 12 | 19 | 19 | 27 | 12 |
| Early stage tumor | 30 | 61 | 16 | 69 | 38 | 34 | 63 | 61 | 57 | 46 | 39 |
| Late stage normal | 38 | 12 | 8 | 37 | 44 | 42 | 43 | 64 | 37 | 38 | 12 |
| Late stage tumor | 15 | 55 | 33 | 46 | 56 | 48 | 17 | 65 | 57 | 20 | 11 |

TABLE 7
Sequencing results for CA4 on cell lines and CRC tumor/adjacent normal tissue
pools at specific CpG dinucleotides

| | CpG sites | | | | | | | | | | |
|---|---|---|---|---|---|---|---|---|---|---|---|
| Sample Type | #6 | #35 | #43 | #52 | #104 | #120 | #127 | #129 | #140 | #153 | #156 |
| HT-29 | 46 | 100 | 88 | 99 | 52 | 82 | 76 | 94 | 88 | 80 | 81 |
| HT-29 treated | 47 | 96 | 77 | 92 | 50 | 67 | 74 | 87 | 84 | 83 | 72 |
| SW480 | 63 | 76 | 65 | 80 | 12 | 91 | 42 | 48 | 43 | 39 | 38 |
| SW480 treated | 70 | 61 | 57 | 73 | 13 | 52 | 44 | 18 | 14 | 17 | 53 |
| SW620 | 70 | 93 | 64 | 91 | 24 | 54 | 52 | 67 | 68 | 43 | 39 |
| SW620 treated | 64 | 89 | 81 | 77 | 33 | 74 | 67 | 80 | 78 | 60 | 69 |
| LS174T | 76 | 35 | 7 | 45 | 15 | 8 | 8 | 25 | 22 | 30 | 43 |
| LS174T treated | 93 | 35 | 39 | 40 | 18 | 22 | 19 | 62 | 28 | 23 | 32 |
| LS123 | 69 | 52 | 56 | 48 | 7 | 15 | 60 | 10 | 54 | 36 | 33 |
| LS123 treated | 75 | 39 | 62 | 69 | 16 | 27 | 70 | 46 | 62 | 43 | 40 |
| Early stage normal | | 58 | 28 | 57 | 41 | 44 | 16 | 1 | 13 | 35 | 1 |
| Early stage tumor | | 95 | 67 | 93 | 52 | 63 | 71 | 80 | 87 | 65 | 82 |
| Late stage normal | | | | 37 | 11 | 15 | 2 | 21 | 15 | 3 | 5 |
| Late stage tumor | | 67 | 55 | 64 | 11 | 17 | 51 | 49 | 69 | 22 | 17 |
| | CpG sites | | | | | | | | | | |
| Sample Type | #158 | #164 | #181 | #190 | #199 | #201 | #204 | #213 | #218 | #220 | #227 |
| HT-29 | 87 | 90 | 66 | 82 | 83 | 100 | 75 | 100 | 66 | 65 | |
| HT-29 treated | 87 | 87 | 73 | 68 | 92 | 100 | 94 | 91 | 65 | 79 | 47 |
| SW480 | 7 | 79 | 63 | 37 | 54 | 79 | 79 | 73 | 78 | 96 | 18 |
| SW480 treated | 7 | 66 | 27 | 57 | 28 | 56 | 27 | 51 | 35 | 32 | 23 |
| SW620 | 53 | 100 | 64 | 32 | 74 | 100 | 100 | 100 | 94 | 100 | 54 |
| SW620 treated | 73 | 92 | 43 | 46 | 10 | 96 | 100 | 96 | 91 | 93 | 37 |
| LS174T | 3 | 68 | 50 | 37 | 11 | 1 | 20 | 35 | 29 | 23 | 9 |
| LS174T treated | 10 | 41 | 22 | 61 | 10 | 67 | 3 | 56 | 64 | 45 | 27 |
| LS123 | 1 | 23 | 21 | 10 | 9 | 2 | 2 | 12 | 14 | 4 | 10 |
| LS123 treated | 22 | 62 | 18 | 11 | 17 | 33 | 20 | 29 | 24 | 20 | 11 |
| Early stage normal | 14 | 29 | 36 | 45 | 33 | 37 | 43 | 66 | 55 | 40 | |
| Early stage tumor | 100 | 90 | 52 | 15 | 89 | 100 | 95 | 98 | 87 | 82 | |
| Late stage normal | 20 | 12 | 15 | | | | | | | | |
| Late stage tumor | 23 | 39 | 20 | | | | | | | | |

TABLE 8
Sequencing results for GPX3 on cell line and CRC tumor/adjacent normal tissue pools at specific CpG dinucleotides

| | CpG sites | | | | | | | | | | | | | |
|---|---|---|---|---|---|---|---|---|---|---|---|---|---|---|
| Sample type | #25 | #27 | #31 | #49 | #56 | #75 | #84 | #86 | #101 | #126 | #129 | #142 | #146 | #167 |
| cell line pool | 83 | 100 | 76 | 80 | 99 | 81 | 82 | 97 | 81 | 83 | 82 | 98 | 81 | 93 |
| treated | 70 | 100 | 67 | 70 | 98 | 72 | 77 | 74 | 84 | 75 | 80 | 97 | 80 | 89 |
| Early stage normal | 37 | 64 | 60 | 51 | 46 | 37 | 45 | | 55 | 32 | 31 | 47 | 54 | 56 |
| Early stage tumor | 63 | 100 | 58 | 68 | 100 | 66 | 73 | | 62 | 75 | 74 | 75 | 74 | 77 |
| Late stage normal | 41 | 65 | 58 | | 37 | 27 | 23 | 50 | | 28 | 40 | 45 | 42 | 41 |
| Late stage tumor | 30 | 59 | 57 | | 17 | 31 | 29 | 56 | | 28 | 45 | 29 | 38 | 36 |

### Example 5

### Verification of Relevant CpG Sites by Methylation-Specific PCR

Samples. Paired tumor and adjacent normal tissue from ten lung cancer and nine colorectal cancer patients was collected under institutional review board (IRB) approval with patient consent. Tissues were flash frozen in LN₂ and stored at −80° C. prior to DNA extraction. Sera from colorectal cancer patients and patients with no evidence of disease were collected under IRB approval and stored at −80° C. prior to DNA purification. All tissues and sera were blinded.

Cell lines. A panel of four lung cancer, five colorectal cancer, one metastatic prostate cancer, and one normal lung fibroblast cell line were amplified for MSP. Five CRC cell lines were treated with the demethylating agent 5-aza-2′-deoxycytidine prior to MSP. Cells were grown to 50% confluence in the appropriate culture medium prior to treatment with 5-aza-2′-deoxycytidine. Optimal concentrations and incubation times (Table 4) were determined by assaying for reduction of p16 promoter methylation using MSP. Cells were harvested, pelleted by centrifugation, and washed twice in Hanks buffered saline solution. Cell pellets were stored at −80° C. Control cells were maintained simultaneously without 5-aza-2′-deocycytidine treatment.

DNA extraction. DNA was purified from tissues and cell lines using the QIAGEN DNeasy® Tissue Kit. Approximately 25-35 mg of each tissue was pulverized under liquid nitrogen before extraction. Elution volume for tissues was 200 μL. A final volume of 200 μL of cell line DNA was extracted from 15 to 25 μL of each packed cell pellet (between 10⁶-10⁷ cells). One mL of each serum DNA was purified with the QIAamp® UltraSens™ Virus Kit. Purified DNA was stored at −20° C.

Bisulfite modification: Modification was performed according to the Frommer method (See Frommer M, et al., PNAS, 89: 1827-1831 (1992).) One μg genomic DNA was diluted into 50 μl with distilled H₂O, 5.5 μl of 2M NaOH was added, and the mixture incubated at 37° C. for 10 minutes (to create single stranded DNA). Thirty μl of freshly prepared 10 mM hydroquinone (Sigma) was added to each tube. Five hundred twenty μl of freshly prepared 3M sodium bisulfite (Sigma S-8890), pH 5.0 was then added. Reagents were thoroughly mixed and then covered with mineral oil and incubated at 50° C. for 16 hours. After removing the oil, 1 ml of Wizard DNA Cleanup Resin (Promega A7280) was added to each tube prior to applying the mixture to miniprep column in the DNA Wizard Cleanup kit. The column was washed with 2 ml of 80% isopropanol, and eluted with 50 μl of heated water (60-70° C.). 5.5 μl of 3 M NaOH to was added to each tube, and incubated at room temperature for 5 minutes. Then 1 μl glycogen was added as carrier, 33 μl of 10 M NH₄Ac, and 3 volumes of ethanol for DNA precipitation. The pellet was spun down and washed with 70% ethanol, dried and resuspended in 20 μl water. In some instances, the EZ DNA Methylation Kit (Zymo Research) which uses a simplified version of the Frommer method was used. In these cases, 1 μg of genomic DNA was denatured in 0.3M NaOH for 15 minutes at 37° C. followed by incubation at 50° C. for 16 hours in 0.5 mM hydroquinone and a saturated solution of sodium bisulfite at pH 5. Modified DNA was bound to the Zymo column membrane, then desulfonated with 0.3M NaOH for 15 minutes at room temperature. DNA was washed and resuspended with 50 μL 10 mM Tris-HCl-0.1 mM EDTA, pH 7.5 and stored at −20° C. The bisulfite reaction results in conversion of an unmethylated cytosine to uracil. Methylated cytosine remains unchanged after the bisulfite reaction. The resulting bisulfite modified DNA is single stranded.

PCR amplification: Primer pairs that discriminate between unmethylated and methylated CpG dinucleotides were designed using Oligo 6 (Molecular Biology Insights, Inc.) (Table 9).

Four μL of modified DNA (1/12 of modification reaction) were amplified in a 16 μL reaction volume containing 10 mM Tris-HCl pH8.3, 50 mM KCl, 1.5 mM to 2 mM MgCl2, (Applied Biosystems), 0.25 mM each dNTP, 0.4 unit AmpliTaq (Applied Biosystems), and MSP primers (each at 200 nM). Cycling conditions were 10 minutes at 95° C., 40 cycles of 30 seconds at 95° C., 30 seconds at 54-62° C., 30 seconds at 72° C., subsequently followed by extension for 5 minutes at 72° C. Amplicons were separated on 3% agarose-1× TBE gels containing ethidium bromide (BioRad Ready Agarose Gels).

TABLE 9
Primers for MSP assays

| Gene | | Forward/ | Primer | Primer Sequences | | Amplicon | SEQ ID |
|---|---|---|---|---|---|---|---|
| name | M/U | reverse | number | 5′-3′ | Tₘ° C. | length | NO |

| | | | | | | | |
|---|---|---|---|---|---|---|---|
| CA4 | M | F | 197 | TCGCGGCGCGGTTATC | 77 | 135 | 269 |
| | | R | 198 | CCACCGACGCTCACCGAT | 77.3 | | 270 |
| CA4 | U | F | 199 | TGGTTTTTTTTGTGGTGTGGTTATT | 73.5 | 149 | 271 |
| | | R | 200 | CAACACCACCAACACTCACCAAT | 75.5 | | 272 |
| SCNN1B | M | F | 201 | TATTCGTGGCGTATGTGGGTATC | 74.1 | 162 | 273 |
| | | R | 202 | ACACGCACGATCCCGACT | 74.4 | | 274 |
| SCNN1B | U | F | 203 | GGATATATTTGTGGTGTATGTGGGTATT | 72.1 | 173 | 275 |
| | | R | 204 | CTAACCACACACACAATCCCAACT | 73.4 | | 276 |
| GPX3 | M | F | 35 | GGTGGGGAGTTGAGGGTAAGTC | 79.2 | 218 | 277 |
| | | R | 36 | CCTACAACAACCGAACCATAACG | 75.5 | | 278 |
| GPX3 | U | F | 39 | GGTGGGGAGTTGAGGGTAAGTT | 77.3 | 220 | 279 |
| | | R | 40 | CACCTACAACAACCAAACCATAACA | 74.1 | | 280 |
| SLC5A8a | M | F | 257 | CGTTTTTTAGGTGTCGGTTTTC | 71.7 | 130 | 281 |
| | | R | 258 | AACAACGAATCGATTTTCCG | 69.1 | | 282 |
| | U | F | 259 | GGTGTTTTTTAGGTGTTGGTTTTT | 70.5 | 134 | 283 |
| | | R | 260 | AAAACAACAAATCAATTTTCCAAA | 65.4 | | 284 |
| SLC5A8b | M | F | 261 | TCGAACGTATTTCGAGGC | 70.4 | 109 | 285 |
| | | R | 262 | ACAACGAATCGATTTTCCG | 68.6 | | 286 |
| | U | F | 263 | TTGAATGTATTTTGAGGTG | 64.3 | 101 | 287 |
| | | R | 264 | TCA ATT TTC CAA AAT CCC | 63.6 | | 288 |
| MLH1 | M | F | 5 | AACGAATTAATAGGAAGAGCGGATAGCG | 77.4 | 164 | 289 |
| | | R | 6 | CGTCCCTCCCTAAAACGACTACTACCC | 81.9 | | 290 |
| MLH1 | U | F | 7 | TAAAAATGAATTAATAGGAAGAGTGGATAGTG | 73.6 | 173 | 291 |
| | | R | 8 | AATCTCTTCATCCCTCCCTAAAACA | 74.1 | | 292 |
| P16 | M | F | 19 | GAGGGTGGGGCGGATCGC | 74.9 | 144 | 293 |
| | | R | 20 | GACCCCGAACCGCGACCG TAA | 78.0 | | 294 |
| P16 | U | F | 21 | TTATTAGAGGGTGGGGTGGATTGT | 70.4 | 150 | 295 |
| | | R | 22 | CAACCCCAAACCACAACCATAA | 73.6 | | 296 |
| MGMT | M | F | 13 | TTTCGACGTTCGTAGGTTTTCGC | 75.5 | 83 | 297 |
| | | R | 14 | GCACTCTTCCGAAAACGAAACG | 75.4 | | 298 |
| MGMT | U | F | 11 | TTTGTGTTTTGATGTTTGTAGGTTTTTGT | 71.7 | 91 | 299 |
| | | R | 12 | AACTCCACACTCTTCCAAAAACAAAACA | 74.5 | | 300 |

In MSP experiments, cell line DNA was used as positive controls for both methylated and unmethylated amplicons for SCNN1B, CA4, and GPX3 (Table 10. Samples for which there was a positive amplicon detected are indicated with at least one “+”. Where no amplicon was seen, there is a “−”. A panel of genes that included SCNN1B, CA4, and CA4 was used to assess the methylation status of 9 additional colorectal cancer and adjacent normal tissues by MSP (Table 11). Differential methylation between tumor and adjacent normal tissue for at least one gene in the panel was shown for 8 of the 9 pairs of samples. Thirty-two serum samples from patients with colorectal cancer were examined by MSP for the presence of methylated amplicon for the genes SCNN1B, CA4, and GPX3. In the serum of six of these patients methylated amplicon was detected (Table 12). All samples had detectable unmethylated sequences for the three genes, reflecting the DNA present in the serum that comes from normal cells. For a set of 10 sera from normal individuals, no methylated sequences were detected.

TABLE 10
Cell lines used as controls in MSP experiments.

| Methylated gene | Cell Line | Primer Numbers | Results |
|---|---|---|---|
| CA4 M | HT29 | 197/198 | + |
| CA4 U | | 199/200 | − |
| CA4 M | SW480 | 197/198 | + |
| CA4 U | | 199/200 | +/− |
| SCNN1B M | SW480 | 201/202 | + |
| SCNN1B U | | 203/204 | + |
| GPX3 M | SW620 | 35/36 | + |
| GPX3 U | | 39/40 | + |
| GPX3 M | HT29 | 35/36 | − |
| GPX3 U | | 39/40 | + |

TABLE 11
Colorectal cancer tissues assessed for methylation using a panel of genes.

| Patient | Dukes | | | | | | |
|---|---|---|---|---|---|---|---|
| ID | stage | SCNN1B | GPX3 | CA4 | p16 | MGMT | hMLH1 |
| 10 | B | + | ++ | + | ++ | − | +/− |
| | | − | + | − | +/− | − | +/− |
| 11 | B | + | ++ | + | + | +++ | +/− |
| | | + | ++ | − | ++ | +++ | +/− |
| 12 | B | − | ++ | +/− | +/− | +/− | + |
| | | − | − | − | − | − | +/− |
| 13 | B | + | ++ | + | − | ++ | + |
| | | − | − | + | +/− | +/− | +/− |
| 14 | B | − | + | + | − | ++ | +/− |
| | | + | + | +/− | + | ++ | +/− |
| 15 | B | − | +/− | − | + | − | + |
| | | − | + | +/− | − | + | +/− |
| 16 | B | + | ++ | − | ++ | ++ | ++ |
| | | + | ++ | − | ++ | ++ | ++ |
| 17 | C | +/− | + | + | + | +/− | +/− |
| | | + | + | − | + | + | + |
| 18 | C | + | + | +/− | + | ++ | + |
| | | − | + | +/− | + | ++ | + |

TABLE 12
Sera from colorectal concer patients with methylated sequences.

| Patient ID | SCNN1B | CA4 | GPX3 |
|---|---|---|---|
| C11 | − | + | − |
| C13 | − | − | + |
| C17 | − | + | − |
| C20 | − | − | + |
| C24 | − | + | − |
| C43 | + | − | − |

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of identifying one or more nucleic acid sequences useful as a biomarker for a disease to be detected, comprising:
(a) identifying one or more nucleic acid sequences that are down-regulated in diseased cells compared to normal cells, wherein the nucleic acid sequences comprise at least one methylated CpG site in a promoter-first exon region;
(b) comparing expression level of the nucleic acid sequences from (a) with expression level of the nucleic acid sequences from (a) that have been demethylated; and
(c) identifying those nucleic acid sequences exhibiting a significant increase in the expression level after demethylation treatment as compared to the expression level of the same nucleic acid sequences in the methylated state.

2. The method of claim 1, wherein the methylation at one or more specific CpG sites of the nucleic acid sequences from (c) in one or more clinical samples obtained from a subject having or suspected of having the disease to be detected, significantly increases over the normal samples.

3. The method of claim 1, wherein the promoter-first exon region spans about 1000 base pairs upstream of the first exon and about 1000 base pairs downstream of the first exon.

4. The method of claim 1, wherein demethylation is accomplished using a bisulfite compound.

5. A method of detecting, the presence or stage of a disease in a subject, comprising:
(a) determining the degree of methylation of one or more CpG sites on nucleic acid sequences in a biological sample obtained from the subject;
(b) determining the presence of, predisposition to, or stage of the disease in the subject based on the degree of methylation.

6. The method of claim 5, wherein the CpG sites are hypermethylated in cells with the disease.

7. The method of claim 5, wherein the disease is cancer.

8. The method of claim 5, wherein the disease is colorectal cancer.

9. The method of claim 5, wherein the nucleic acid sequences are the ones identified by the method of claim 1.

10. The method of claim 5, wherein the nucleic acid sequences are the ones identified by the method of claim 2.

11. The method of claim 5, wherein the nucleic acid sequences comprise a nucleic acid sequence selected from the group consisting of SEQ ID Nos: 1-129.

12. A method of monitoring the onset, progression, or regression of a disease in a subject, comprising:
(a) detecting in a biological sample of the subject at a first point in time, the degree of methylation of one or more CpG sites on nucleic acid sequences, wherein the CpG sites are differentially methylated at different stages of the disease;
(b) repeating step (a) at a subsequent point in time; and
(c) comparing the degree of methylation of the CpG sites in step (a) and (b), wherein a change in the degree of methylation is indicative of disease progression in the subject.

13. The method of claim 12, wherein the CpG sites are hypermethylated in cells with the disease.

14. The method of claim 12, wherein the disease is cancer.

15. The method of claim 12, wherein the disease is colorectal cancer.

16. The method of claim 12, wherein the CpG sites are the ones identified by the method of claim 1.

17. The method of claim 12, wherein the nucleic acid sequences are the ones identified by the method of claim 2.

18. The method of claim 12, wherein the nucleic acid sequences comprise a nucleic acid sequence selected from the group consisting of SEQ ID Nos: 1-129.

19. A method of determining the efficacy of a test compound for inhibiting a disease in a subject, comprising:
(a) detecting in a first biological sample of the subject, the degree of methylation of one or more CpG sites, wherein the sample has not been exposed to the test compound, and wherein the CpG sites are methylated in the disease;
(b) detecting in a second biological sample of the subject, the degree of methylation of the same CpG sites, wherein the sample has been exposed to the test compound; and
(c) comparing the degree of methylation of the CpG sites in step (a) and (b), wherein a decrease in methylation after the sample has been exposed to the test compound, is indicative of the efficacy of the test compound.

20. The method of claim 19, wherein the CpG sites are hypermethylated in cells with the disease.

21. The method of claim 19, wherein the disease is cancer.

22. The method of claim 19, wherein the disease is colorectal cancer.

23. The method of claim 19, wherein the CpG sites are the ones identified by the method of claim 1.

24. The method of claim 19, wherein the nucleic acid sequences are the ones identified by the method of claim 2.

25. The method of claim 19, wherein the nucleic acid sequences comprise a nucleic acid sequence selected from the group consisting of SEQ ID Nos: 1-129.

26. A kit useful for diagnosis, prognosis, staging, monitoring, and therapeutic treatment of a disease, comprising a bisulfite reagent, and one or more nucleic acid molecules comprising at least about 9 consecutive nucleotides in length that is specific for detecting methylation of one or more CpG sites on one or more nucleic acid marker sequences.

27. The kit of claim 26, further comprising one or more primers and probes comprising at least about 9 consecutive nucleotides in length that is specific for detecting the expression levels of said nucleic acid marker sequences.
